(19) United States
(12) Patent Application Publication
(10) Pub. No.: US 2022/0409745 A1
(43) Pub. Date: Dec. 29, 2022

(54) COMPOSITIONS AND METHODS OF USING ENGINEERED FUSION PROTEINS THAT BIND G4C2 HUMAN REPEATS

(71) Applicant: The Regents of the University of California, Oakland, CA (US)

(72) Inventors: Eugene Yeo, La Jolla, CA (US); Kathryn H. Morelli, La Jolla, CA (US)

(21) Appl. No.: 17/777,741

(22) PCT Filed: Nov. 18, 2020
(86) PCT No.: PCT/US20/61033

§ 371 Date: May 18, 2022

Related U.S. Application Data
(60) Provisional application No. 62/937,468, filed on Nov. 19, 2019.
(60) Provisional application No. 62/952,744, filed on Dec. 23, 2019.

(51) Int. Cl. A61K 48/00; C12N 15/86; C12N 15/62; C12N 9/22; C07K 14/47; A61P 25/00

(52) U.S. Cl. CPC A61K 48/005; C12N 15/86; C12N 15/62; C12N 9/22; C07K 14/47; A61P 25/00; C12N 2750/14143; C07K 2319/00

(57) ABSTRACT

Provided herein are fusion proteins, isolated nucleic acids encoding a fusion protein, and gene delivery vectors comprising the same, wherein the isolated nucleic acids comprise: (i) a first sequence encoding a RNA-binding zinc finger domain; and (ii) a second sequence encoding a fusion partner; and methods of using the same.

### CROSS-REFERENCE TO RELATED APPLICATIONS

This application claims priority to U.S. Provisional Patent Application No. 62/937,468, filed Nov. 19, 2019 and U.S. Provisional Patent Application No. 62/952,744, filed Dec. 23, 2019. The contents of each of these applications are incorporated herein by reference in their entireties.

### TECHNICAL FIELD

The present disclosure relates to methods and materials for treating a patient having a disease associated with G₄C₂ hexanucleotide repeat expansions.

### BACKGROUND

Zinc-finger (ZNF) domain containing proteins (ZNPs) are one of the most diverse and numerous groups of proteins. The zinc-finger motifs within these proteins are maintained by a zinc ion, which coordinates cysteine and histidine in different combinations allowing ZNFs to have the ability to interact with and direct changes in DNA and/or RNA (Radecke et al., Mol. Ther., 18(4):743-753 (2010); Jabalameli et al., Gene, 558(1):1-5 (2015)). Recently, a mouse zinc finger protein, Zpf106, has been identified to specifically bind a hexanucleotide repeat expansion, G₄C₂, in RNA (Celona et al., Elife, 10(6): pii: e19032. doi: 10.7554/eLife.19032 (2017); Anderson et al., Proc. Natl. Aca. Sci., 113(31):E4494-503 (2016)). Naturally occurring G₄C₂ hexanucleotide repeat expansions within C9ORF72 transcripts have been identified to cause the most common form of familial Amyotrophic Lateral Sclerosis as well as Frontal Temporal Dementia.

### SUMMARY

This document provides compositions, methods, and material for treating a patient having a disease associated with G₄C₂ hexanucleotide repeat expansions. For example, provided herein are isolated nucleic acids encoding a fusion protein and gene delivery vectors comprising same, where the isolated nucleic acids included: (i) a first sequence encoding a RNA-binding zinc finger domain; and (ii) a second sequence encoding a fusion partner; and methods of using the same. The present disclosure is based on the discovery that RNA targeting ZNF motifs from the human ortholog of Zpf106, known as ZNF106, can serve as a surrogate RNA-binding motif that can be used to direct proteins (e.g., reporters or RNA degrading enzymes) to human RNA transcripts that contain expanded G₄C₂ hexanucleotide repeats. In addition, the present disclosure is based on the discovery that RNA targeting ZNF motifs from ZNF106 can be used to direct proteins (e.g., reporters or RNA degrading enzymes) to human RNA transcripts that contain expanded C₄G₂ hexanucleotide repeats.

In one aspect, this disclosure features isolated nucleic acids encoding a fusion protein, wherein the isolated nucleic acid includes: (i) a first sequence encoding a RNA-binding zinc finger domain or a fragment thereof including: an amino acid sequence that is at least 90% identical to the sequence of HECRVCGVTEVGLSAYAKHISGQLH (SEQ ID NO: 1), or an amino acid sequence that is at least 90% identical to the sequence of YRCWWHGCSLIFGVVDHLKQHLLTDH (SEQ ID NO: 2); and (ii) a second sequence encoding a fusion partner.

In some embodiments, the first sequence further includes an amino acid sequence encoding a second RNA-binding zinc finger domain. In some embodiments where the first sequence includes a first RNA-binding zinc finger domain and a second RNA-binding zinc finger domain, (i) the first RNA-binding zinc finger domain includes SEQ ID NO: 1 and the second RNA-binding zinc finger domain includes SEQ ID NO: 2; (ii) the first RNA-binding zinc finger domain includes SEQ ID NO: 2 and the second RNA-binding zinc finger domain includes SEQ ID NO: 1; (iii) the first RNA-binding zinc finger domain includes SEQ ID NO: 1 and the second RNA-binding zinc finger domain includes SEQ ID NO: 1; or (iv) the first RNA-binding zinc finger domain includes SEQ ID NO: 2 and the second RNA-binding zinc finger domain includes SEQ ID NO: 2. In some embodiments, the first RNA-binding zinc finger domain is directly adjacent to the second RNA-binding zinc finger domain. In some embodiments, the isolated nucleic acid further includes a sequence encoding a linker positioned between the first RNA-binding zinc finger domain and the second RNA-binding zinc finger domain.

In some embodiments, the first sequence encoding the RNA-binding zinc finger domain includes three or more RNA-binding zinc finger domains.

In some embodiments of the isolated nucleic acid sequence, the first sequence is directly adjacent to the second sequence. In some embodiments, the isolated nucleic acid further includes a sequence encoding a linker positioned between the first sequence and the second sequence.

In some embodiments, the fusion partner includes a RNA degrading enzyme. In some embodiments, the RNA degrading enzyme includes an endonuclease, a 5′ exonuclease, or a 3′ exonuclease. In some embodiments, the endonuclease includes a human endonuclease, wherein the human endonuclease cleaves single stranded RNA. In some embodiments, the endonuclease includes a PIN (PilT N-terminal domain) RNA endonuclease domain or active fragment thereof.

In another aspect, this disclosure features gene delivery vectors including any of the isolated nucleic acid sequences described herein. In some embodiments, the gene delivery vector is selected from the group consisting of an adenoviral vector, an adeno associated viral (AAV) vector, a lentiviral vector, and a retroviral vector. In some embodiments, the gene delivery vector is an AAV9 vector.

In another aspect, this disclosure features pharmaceutical compositions including any of the isolated nucleic acids described herein or any of the gene delivery vectors described herein.

In another aspect, this disclosure features methods of decreasing a level of RNA having a G₄C₂ hexanucleotide repeat in the central nervous system (CNS) of a subject in need thereof, including administering to the subject an effective amount of any of the isolated nucleic acids described herein, any of the gene delivery vectors described herein or any of the pharmaceutical compositions described herein.

In another aspect, this disclosure features methods of treating a subject having a G₄C₂ or C₄G₂ hexanucleotide repeat-associated disease or disorder including administering to the subject a therapeutically effective amount of any of the isolated nucleic acids described herein, any of the gene delivery vectors described herein or any of the pharmaceutical compositions described herein.

In some embodiments, the subject is previously diagnosed or identified as having a G₄C₂ or a C₄G₂ hexanucleotide repeat-associated disease or disorder. In some embodiments, the G₄C₂ hexanucleotide repeat-associated disease or disorder is frontotemporal dementia (FTD) or amyotrophic lateral sclerosis (ALS).

Unless otherwise defined, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the disclosure will be apparent from the following detailed description, and from the claims.

### BRIEF DESCRIPTION OF DRAWINGS

FIG. 1 is a schematic diagram showing the basic protein structure of human ZFP106. ZFP106 contains two C2H2 zinc finger domains (Znf1 and Znf2), a nuclear localization signal (NLS) and WD40 repeat domains.

FIG. 2 is a gel image of an electrophoretic mobility-shift assay (EMSA) assay showing purified Znf1 binds to both G-q and non-G-q structures (G₄C₂)₈.

FIG. 3A is a RNA (Northern) dot blot of (G₄C₂) expression levels in COS-M6 cells transfected with (G₄C₂)₆₆, G₄C₂-targeting ZFP AAVs (AAV-Znf1, AAV-Znf2, or AAV-Znf1+2), or a non-targeting AAV construct expressing GFP (AAV-GFP). U6 snRNA served as a loading control.

FIG. 3B is a histogram showing quantification of the RNA (Northern) dot blot in FIG. 3A. Data was normalized to U6 and background was subtracted.

FIG. 4A is a RNA (Northern) dot blot of (C₄G₂) expression levels in COS-M6 cells transfected with (C₄G₂)₁₀₅ RNA, C₄G₂-targeting ZFP AAVs (AAV-Znf1, AAV-Znf2, AAV-Znf1+2) or a non-targeting AAV construct (non-targeting PIN). U6 snRNA served as a loading control.

FIG. 4B is a histogram showing quantification of the RNA (Northern) dot blot in FIG. 4A. Data was normalized to U6 and background was subtracted.

FIG. 5A is a RNA (Northern) dot blot of (CUG) expression levels in COS-M6 cells transfected with (CUG)₁₀₅, G₄C₂+C₂G₄-targeting ZFP AAV-Znf1 (AAV-Z1) or a non-targeting AAV construct (non-targeting PIN).

FIG. 5B is a RNA (Northern) dot blot of (CAG) expression levels in COS-M6 cells transfected with (CAG)₁₀₅ and a G₄C₂+C₂G₄-targeting ZFP AAV-Znf1 (AAV-Z1) or a non-targeting AAV construct (non-targeting PIN).

FIG. 6 is a panel of immunofluorescence images of spinal organoids. Left panel: image taken at 14 days of differentiation with neural progenitors identified as PAX6⁺ and Nestin⁺. Middle panel: image taken at day 30 (1 month) at around the time point when neural progenitors start to differentiate into interneuron and oligodendrocyte precursors as identified by NKX2.2⁺. Right panel: image taken at 60 days (2 months) around the time point when precursors from middle panel eventually develop into mature motor neurons as identified by Islet1⁺ cells.

FIG. 7A shows immunofluorescence images of spinal organoids stained for HA-tagged Z1-PIN and GFAP.

FIG. 7B is a histogram showing quantification of cells positive for Z1 expression from the images in FIG. 7A.

FIG. 8A is a panel of fluorescent images from a fluorescent in situ hybridization (FISH) assay. White arrow points to a sense FISH probe hybridized to the antisense C₄G₂ repeat within C9-ALS patient iPSC-derived spinal organoids transduced with (right panel) or without (left panel) scAAV9-Z1.

FIG. 8B is a histogram showing quantification of the percentage of cells with antisense C₄G₂ foci in C9-ALS spinal organoids transduced with (Z1-treated) or without (untreated) scAAV9-Z1 as compared to non-disease (control) spinal organoids transduced with (Z1-treated) and without (untreated) scAAV9-Z1.

FIG. 9A is a panel of fluorescent images from a fluorescent in situ hybridization (FISH) assay. White arrow points to an antisense FISH probe hybridized to the sense G₄C₂ repeat within C9-ALS patient iPSC-derived spinal organoids transduced with (right panel) or without (left panel) scAAV9-Z1.

FIG. 9B is a histogram showing quantification of the percentage of cells with sense G₄C₂ foci in C9-ALS spinal organoids transduced with (Z1-treated) or without (untreated) scAAV9-Z1 as compared to non-disease (control) spinal organoids transduced with (Z1-treated) and without (untreated) scAAV9-Z1.

### DETAILED DESCRIPTION

This document provides isolated nucleic acids encoding a fusion protein, wherein the isolated nucleic acid includes: (i) a first sequence encoding a RNA-binding zinc finger domain (e.g., any of the exemplary RNA-binding zinc finger domain described herein); and (ii) a second sequence encoding a fusion partner (e.g., any of the exemplary fusion partners described herein or known in the art). Also provided are gene delivery vectors that include any of the isolated nucleic acids provided herein, pharmaceutical compositions including any of the gene delivery vectors described herein, and kits including any of the pharmaceutical compositions described herein. Also provided herein are mammalian cells (e.g., any of the exemplary mammalian cells described herein) transfected with any of the isolated nucleic acids described herein or transduced with any of the gene delivery vectors described herein.

Also provided herein are heterologous fusion proteins that include: (i) a RNA-binding zinc finger domain (e.g., any of the exemplary RNA-binding zinc finger domain described herein); and (ii) a fusion partner (e.g., any of the exemplary fusion partners described herein or known in the art). Also provided are pharmaceutical compositions including any of the heterologous fusion proteins described herein, and kits including any of the same.

Also provided herein are methods decreasing a level of RNA having a G₄C₂ hexanucleotide repeat in the central nervous system (CNS) of a subject in need thereof that include administering to the subject any of the gene delivery vectors described herein, any of the fusion proteins described herein, or any of the pharmaceutical compositions described herein.

Also provided herein are methods for treating a subject having a disease associated with G₄C₂ hexanucleotide expanded repeats administering to the subject a therapeutically effective amount of any of the gene delivery vectors described herein, any of the fusion proteins described herein, or any of the pharmaceutical compositions described herein. Additional non-limiting aspects of the gene delivery vectors, fusion proteins, pharmaceutical compositions, kits, and methods are described herein and can be used in any combination without limitation.

Also provided herein are methods of tracking a RNA having a G₄C₂ hexanucleotide repeat or measuring the amount of a RNA having a G₄C₂ hexanucleotide repeat in a cell where the includes administering to a cell any of the isolated nucleic acid sequences described herein or any the gene delivery vehicle described herein. In some embodiments, the fusion protein includes a reporter where the fusion protein binds to RNA having the G₄C₂ hexanucleotide repeat in the cell and the reporter is used to determine the location of the RNA having the G₄C₂ hexanucleotide repeat and/or determining the amount of RNA having the G₄C₂ hexanucleotide repeat based on detection of the reporter.

Also provided herein are methods decreasing a level of RNA having a C₄G₂ hexanucleotide repeat in the central nervous system (CNS) of a subject in need thereof that include administering to the subject any of the gene delivery vectors described herein, any of the fusion proteins described herein, or any of the pharmaceutical compositions described herein.

Also provided herein are methods for treating a subject having a disease associated with C₄G₂ hexanucleotide expanded repeats administering to the subject a therapeutically effective amount of any of the gene delivery vectors described herein, any of the fusion proteins described herein, or any of the pharmaceutical compositions described herein.

Also provided herein are methods of tracking an RNA having a C₄G₂ hexanucleotide repeat or measuring the amount of an RNA having a C₄G₂ hexanucleotide repeat in a cell where the includes administering to a cell any of the isolated nucleic acid sequences described herein or any the gene delivery vehicle described herein. In some embodiments, the fusion protein includes a reporter where the fusion protein binds to RNA having the C₄G₂ hexanucleotide repeat in the cell and the reporter is used to determine the location of the RNA having the C₄G₂ hexanucleotide repeat and/or determining the amount of RNA having the C₄G₂ hexanucleotide repeat based on detection of the reporter.

The term “a” and “an” refers to one or more (i.e., at least one) of the grammatical object of the article. By way of example, “a cell” encompasses one or more cells.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

Unless otherwise specified, a “nucleotide sequence encoding a protein” includes all nucleotide sequences that are degenerate versions of each other and thus encode the same amino acid sequence.

The term “exogenous” refers to any material introduced from or originating from outside a cell, a tissue or an organism that is not produced by or does not originate from the same cell, tissue, or organism in which it is being introduced.

The term “transduced”, “transfected”, or “transformed” refers to a process by which exogenous nucleic acid is introduced or transferred into a cell. A “transduced,” “transfected,” or “transformed” mammalian cell is one that has been transduced, transfected or transformed with exogenous nucleic acid (e.g., a gene delivery vector) that includes an exogenous nucleic acid encoding RNA-binding zinc finger domain).

The term “subject” is intended to include any mammal. In some embodiments, the subject is cat, a dog, a goat, a human, a non-human primate, a rodent (e.g., a mouse or a rat), a pig, or a sheep. In some embodiments, the subject has or is at risk of developing a CNS disorder or disease. In some embodiments, the subject has previously been identified or diagnosed as having a CNS disorder or disease.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

The term “RNA having a G₄C₂ hexanucleotide expanded repeat” or “RNA having a G₄C₂ hexanucleotide repeat expansion” refers to a RNA having greater than about 10 (e.g., greater than about 20, greater than about 30, greater than about 40, greater than about 50, greater than about 60, greater than about 70, greater than about 80, greater than about 90, or greater than about 100) hexanucleotide GGGGCC (G₄C₂) repeats.

The term “RNA having a C₄G₂ hexanucleotide expanded repeat” or “RNA having a C₄G₂ hexanucleotide repeat expansion” can refer to an RNA having greater than about 10 (e.g., greater than about 20, greater than about 30, greater than about 40, greater than about 50, greater than about 60, greater than about 70, greater than about 80, greater than about 90, greater than about 100, greater than about 110, greater than about 120, greater than about 130, greater than about 140, or greater than about 150) hexanucleotide CCCCGG (C₄G₂) repeats.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

The term “treating” means a reduction in the number, frequency, severity, or duration of one or more (e.g., two, three, four, five, or six) symptoms of a disease or disorder in a subject (e.g., any of the subjects described herein), and/or results in a decrease in the development and/or worsening of one or more symptoms of a disease or disorder in a subject.

The term “a G₄C₂ hexanucleotide repeat-associated disease” means a condition that is caused, at least in part, by a RNA having G₄C₂ hexanucleotide repeat expansion in a subject as compared to a subject not having a RNA with G₄C₂ hexanucleotide repeat expansion. Non-limiting examples of a G₄C₂ hexanucleotide repeat-associated disease or disorder is frontotemporal dementia (FTD) or amyotrophic lateral sclerosis (ALS).

The term “a C₄G₂ hexanucleotide repeat-associated disease” can mean a condition that is caused, at least in part, by a RNA having C₄G₂ hexanucleotide repeat expansion in a subject as compared to a subject not having a RNA with C₄G₂ hexanucleotide repeat expansion. Non-limiting examples of a C₄G₂ hexanucleotide repeat-associated disease or disorder is frontotemporal dementia (FTD) or amyotrophic lateral sclerosis (ALS). The term “administer” refers to a method of delivering agents, compounds, or compositions to the desired site of biological action. These methods include, but are not limited to, topical delivery, parenteral delivery, intravenous delivery, intradermal delivery, intramuscular delivery, colonic delivery, rectal delivery, or intraperitoneal delivery. In one embodiment, the compositions described herein are administered intravenously.

The term “promoter” means a DNA sequence recognized by enzymes/proteins in a mammalian cell required to initiate the transcription of an operably linked coding sequence (e.g., a nucleic acid encoding a fusion protein (e.g., a RNA-binding zinc finger domain and a fusion partner)). A promoter typically refers, to e.g. a nucleotide sequence to which an RNA polymerase and/or any associated factor binds and at which transcription is initiated. The promoter can be constitutive, inducible, or tissue-specific (e.g., a brain-specific promoter).

Non-limiting examples of CNS-specific promoters are known in the art (see e.g., Portales-Casamer et al., PNAS, 38:16589-16594 (2010)).

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window or designated region, as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

### Isolated Nucleic Acids

Provided herein are isolated nucleic acids that encode a fusion protein, where the isolated nucleic acid includes: (i) a first sequence encoding a RNA-binding zinc finger domain or a fragment thereof (e.g., any of the RNA-binding zinc finger domain or fragments described herein); and (ii) a second sequence encoding a fusion partner (e.g., any of the exemplary fusion partners described herein). The terms “fusion”, “fused”, and “fusing”, are used herein as is known and applied in the art. In some embodiments, nucleic acids including two or more genes that typically code separate proteins can be positioned in relation to each other to encode a fusion protein. In some embodiments, a nucleic acid encoding a RNA-binding zinc finger domain and a nucleic acid encoding a fusion partner (e.g., a RNA degrading enzyme) can be positioned in relation to each other to encode a fusion protein. In some embodiments, a nucleic acid encoding a first RNA-binding zinc finger domain, a nucleic acid encoding a second RNA-binding zinc finger domain, and a nucleic acid encoding a fusion partner (e.g., a RNA degrading enzyme) can be positioned in relation to each other to encode a fusion protein. In some embodiments, the two or more genes can be positioned in relation to each other using a linker sequence. In some embodiments, the two or more genes encode two or more polypeptides fused together by way of a linker sequence.

In some embodiments, the RNA-binding zinc finger domain includes a first RNA-binding zinc finger domain (e.g., any of the exemplary RNA-binding zinc finger domains) and a second RNA-binding zinc finger domain (e.g., any of the exemplary RNA-binding zinc finger domains). In some embodiments, the RNA-binding zinc finger domain includes three or more, four or more, five or more, or six or more RNA-binding zinc finger domains.

In some embodiments, the fusion partner includes a reporter and/or a RNA degrading enzyme. For example, the fusion partner can be a RNA degrading enzyme. In another example, the fusion partner can be a reporter. In some embodiments, the fusion partner includes a first sequence encoding a first fusion partner (e.g., any of the exemplary fusion partners described herein) and a second sequence encoding a second fusion partner (e.g., any of the exemplary fusion partners described herein).

In some embodiments of any of the isolated nucleic acid sequences, the first sequence and the second sequence are operably linked to a promoter.

In some embodiments of these nucleic acids, the first sequence is positioned 5′ relative to the second sequence in the isolated nucleic acid sequence.

In some examples of any of the isolated nucleic acids, the isolated nucleic acid further includes a sequence encoding a linker (e.g., any of the exemplary linkers described herein or known in the art) positioned between the first sequence and the second sequence.

In some embodiments, the linker can be (G₄S)ₙ (SEQ ID NO: 33), wherein n is an integer between 1 and 10. In some embodiments, the linker includes a (G₄S)ₙ, wherein n is 1, 2, 3, 4, or 5. In some embodiments, the linker can be a XTEN linker including a SEQ ID NO: 30-32.

In some examples of any of the isolated nucleic acids, the first sequence and the second sequence are directly adjacent to each other in the isolated nucleic acid.

In some examples of any of the isolated nucleic acids described herein, the fusion protein encoded by any of the isolated nucleic acids described herein includes a total of about 400 amino acids to about 1,000 amino acids, about 400 amino acids to about 900 amino acids, about 400 amino acids to about 800 amino acids, about 400 amino acids to about 700 amino acids, about 400 amino acids to about 600 amino acids, about 400 amino acids to about 500 amino acids, about 500 amino acids to about 1,000 amino acids, about 500 amino acids to about 900 amino acids, about 500 amino acids to about 800 amino acids, about 500 amino acids to about 700 amino acids, about 500 amino acids to about 600 amino acids, about 600 amino acids to about 1,000 amino acids, about 600 amino acids to about 900 amino acids, about 600 amino acids to about 800 amino acids, about 600 amino acids to about 700 amino acids, about 700 amino acids to about 1,000 amino acids, about 700 amino acids to about 900 amino acids, about 700 amino acids to about 800 amino acids, about 800 amino acids to about 1,000 amino acids, about 800 amino acids to about 900 amino acids, or about 900 amino acids to about 1,000 amino acids.

In some embodiments of any of the isolated nucleic acids, the isolated nucleic acid that encodes any of the fusion proteins described herein includes a total of about 1,200 to about 3,000 nucleotides, about 1,200 to about 2,700 nucleotides, about 1,200 to about 2,400 nucleotides, about 1,200 to about 2,100 nucleotides, about 1,200 to about 1,800 nucleotides, about 1,200 to about 1,500 nucleotides, about 1,500 to about 3,000 nucleotides, about 1,500 to about 2,700 nucleotides, about 1,500 to about 2,400 nucleotides, about 1,500 to about 2,100 nucleotides, about 1,500 to about 1,800 nucleotides, about 1,800 to about 3,000 nucleotides, about 1,800 to about 2,700 nucleotides, about 1,800 to about 2,400 nucleotides, about 1,800 to about 2,100 nucleotides, about 2,100 to about 3,000 nucleotides, about 2,100 to about 2,700 nucleotides, about 2,100 to about 2,400 nucleotides, about 2,400 to about 3,000 nucleotides, about 2,400 to about 2,700 nucleotides, or about 2,700 to about 3,000 nucleotides.

### RNA-Binding Zinc Finger Domains

The mouse zinc finger protein 106, ZFP106, encodes zinc finger motifs (Znf1 and Znf2) that specifically bind a hexanucleotide repeat expansion, G₄C₂, in RNA. The human ortholog of Zpf106, known as ZNF106, can serve as a surrogate RNA-binding zinc finger domain that can be used to direct human proteins to human RNA transcripts that contain expanded G₄C₂ hexanucleotide repeats. The basic protein structure of human ZFP106 and location of the two C2H2 zinc finger domains (Znf1 and Znf2). Znf1 is located at amino acid (aa) position 20-44 while Znf2 is located at aa 1813-1838. Naturally occurring G₄C₂ hexanucleotide repeat expansions within RNA transcripts (e.g., C9ORF72) have been identified to cause the most common form of familial Amyotrophic Lateral Sclerosis as well as Frontal Temporal Dementia.

The mouse zinc finger protein 106, ZFP106, encoding zinc finger motif Znf1 specifically bind a hexanucleotide repeat expansion, C₄G₂, in RNA. The human ZNF1 of the human ZNF1-6 ortholog can serve as a surrogate RNA-binding zinc finger domain that can be used to direct human proteins to human RNA transcripts that contain expanded C₄G₂ hexanucleotide repeats. Accumulation of toxic dipeptide repeat proteins can result from repeat-associated non-ATG (RAN) translation from the expanded antisense, C₄G₂, strand of the G₄C₂ RNA. The C₄G₂ hexanucleotide repeat expansions within RNA transcripts (e.g., C9ORF72) contribute to the RNA toxicity associated with the most common form of familial Amyotrophic Lateral Sclerosis as well as Frontal Temporal Dementia.

Exemplary RNA-binding zinc finger domains can include an amino acid sequence that is at least 70% identical (e.g., at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical) to SEQ ID NO: 1. Additional exemplary RNA-binding zinc finger domains can include an amino acid sequence that is at least 80% identical (e.g., at least 82%, at least 84%, at least 86%, at least 88%, at least 90%, at least 92%, at least 94%, at least 96%, at least 98%, at least 99%, or 100% identical) to SEQ ID NO: 2. In some embodiments, the RNA-binding zinc finger domains of SEQ ID NO: 1 and SEQ ID NO: 2 bind to RNA having a C₄G₂ hexanucleotide repeat. In some embodiments, the RNA-binding zinc finger domains of SEQ ID NO: 1 and SEQ ID NO: 2 bind to RNA having a G₄C₂ hexanucleotide repeat. In some embodiments, a RNA-binding zinc finger domain can be referred to as binding to both a C₄G₂ hexanucleotide repeat and a G₄C₂ hexanucleotide repeat.

In some embodiments, the RNA-binding zinc finger domain is encoded by a nucleic acid sequence that is at least 70% identical (e.g., at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical) to SEQ ID NO: 3. In some embodiments, the RNA-binding zinc finger domain is encoded by a nucleic acid sequence that is at least 70% identical (e.g., at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical) to SEQ ID NO: 4.

As one skilled in the art can appreciate, mutation of an amino acid that is not conserved between different mammalian species is less likely to negatively alter the activity of a protein (e.g., RNA-binding zinc finger domain), while mutation of an amino acid that is conserved between mammalian species is more likely to negatively alter the activity of a protein (e.g., RNA-binding zinc finger domain). Methods of introducing one or more amino acid substitutions into a RNA-binding zinc finger domains are known in the art.

In some embodiments, the RNA-binding zinc finger domain includes a first RNA-binding zinc finger domain (e.g., any of the exemplary RNA-binding zinc finger domains) and a second RNA-binding zinc finger domain (e.g., any of the exemplary RNA-binding zinc finger domains). For example, the RNA-binding zinc finger domain includes a first RNA-binding zinc finger domain including a sequence of SEQ ID NO: 1 or SEQ ID NO: 2 and a second RNA-binding zinc finger domain including a sequence of SEQ ID NO: 1 or SEQ ID NO: 2. In some cases, the RNA-binding zinc finger domain includes a first RNA-binding zinc finger domain including a sequence of SEQ ID NO: 1 and a second RNA-binding zinc finger domain including a sequence of SEQ ID NO: 2. In some cases, the RNA-binding zinc finger domain includes a first RNA-binding zinc finger domain including a sequence of SEQ ID NO: 2 and a second RNA-binding zinc finger domain including a sequence of SEQ ID NO: 1. In some cases, the RNA-binding zinc finger domain includes a first RNA-binding zinc finger domain including a sequence of SEQ ID NO: 1 and a second RNA-binding zinc finger domain including a sequence of SEQ ID NO: 1. In some cases, the RNA-binding zinc finger domain includes a first RNA-binding zinc finger domain including a sequence of SEQ ID NO: 2 and a second RNA-binding zinc finger domain including a sequence of SEQ ID NO: 2. In some embodiments, the RNA-binding zinc finger domain comprises three or more, four or more, five or more, or six or more RNA-binding zinc finger domains.

In some embodiments where the RNA-binding zinc finger domain includes a first RNA-binding zinc finger domain (e.g., any of the exemplary RNA-binding zinc finger domains described herein) and a second RNA-binding zinc finger domain (e.g., any of the exemplary RNA-binding zinc finger domains described herein), the first RNA-binding zinc finger domain is directly adjacent to the second RNA-binding zinc finger domain.

In some embodiments where the RNA-binding zinc finger domain includes a first RNA-binding zinc finger domain (e.g., any of the exemplary RNA-binding zinc finger domains described herein) and a second RNA-binding zinc finger domain (e.g., any of the exemplary RNA-binding zinc finger domains described herein), a linker is positioned between the first RNA-binding zinc finger domain and the second RNA-binding zinc finger domain. In some embodiments, the linker includes about 1 amino acid to about 20 amino acids (or any of the subranges described herein). Non-limiting examples of linkers include linkers having the amino acid sequence (G4S)ₙ (SEQ ID NO: 33) where n is 1, 2, 3, 4, or 5.

In some embodiments, RNA-binding zinc finger domains are fused to a reporter sequence. Non-limiting examples of reporter sequences that can be fused to a RNA-binding zinc finger domain include a human influenza hemagglutinin (HA)-tag, a FLAG™ tag, a HIS-tag (e.g., a hexa histidine-tag). For example, a RNA-binding zinc finger domain (e.g., any of the exemplary RNA-binding zinc finger domains described herein) can be fused to a HA-Tag. The reporter sequence can be fused to either the 5′ or 3′ end of the RNA-binding zinc finger domain. In some embodiments, the RNA-binding zinc finger domain and the reporter sequence are directly adjacent to each other. In some embodiments, a linker (e.g., any of the exemplary linkers described herein) is disposed between the RNA-binding zinc finger domain and the reporter sequence.

In some embodiments, the RNA-binding zinc finger domain that includes an HA-tag and one or more RNA-binding zinc finger domains is encoded by a nucleic acid sequence that is at least 70% identical (e.g., at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical) to SEQ ID NOs: 5-10.

### Fusion Partners

Provided herein are nucleic acid sequences where the second sequence encodes a fusion partner. In some embodiments, the fusion partner includes a reporter and/or a RNA degrading enzyme.

In some embodiments, the fusion partner includes a RNA degrading enzyme. Non-limiting examples of an RNA-degrading enzymes include endonucleases, a 5′ exonucleases, or a 3′ exonucleases. In some embodiments, the fusion partner includes a sequence encoding a human endonuclease, wherein the endonuclease cleaves single stranded RNA. In some embodiments, the endonuclease includes a PIN (PilT N-terminal domain) RNA endonuclease domain or active fragment thereof.

In some embodiments, the fusion partner includes a reporter. Non-limiting examples of reporter sequences include nucleic acid sequences encoding a human influenza hemagglutinin (HA)-tag, a FLAG™ tag, a HIS-tag (e.g., a hexa histidine-tag), a beta-lactamase, a fluorescent protein (e.g., a green fluorescent protein (GFP) or a red fluorescent protein (RFP)), and a luminescent protein (e.g., a luciferase). Additional examples of reporter sequences are known in the art. A reporter can be detected by conventional means, including colorimetric, enzymatic, fluorescence, radiographic, or other spectrographic assays, such as immunological assays (e.g., enzyme-linked immunosorbent assay (ELISA), immunohistochemistry, and radioimmunoassay (RIA)), and fluorescent activating cell sorting (FACS) assays.

In some embodiments, the fusion partner includes a sequence encoding a first fusion partner (e.g., any of the exemplary fusion partners described herein) and a sequence encoding a second fusion partner (e.g., any of the exemplary fusion partners described herein). In some embodiments where the fusion partner includes a first fusion partner and a second fusion partner, the first fusion partner is a reporter sequence (e.g., any of the exemplary reporter sequences described herein or known in the art) and the second fusion partner is a RNA degrading enzyme (e.g., any of the RNA degrading enzymes described herein). For example, a fusion partner can include a first sequence encoding a first fusion partner encoding a HA-Tag and a second fusion partner encoding a PIN domain RNA endonuclease. In some embodiments, the fusion partner includes a sequence encoding a linker sequence (e.g., any of the exemplary linkers described herein) between the first fusion partner (e.g., any of the exemplary fusion partners described herein) and the second fusion partner (e.g., any of the exemplary fusion partners described herein). In some embodiments, the first fusion partner and second fusion partner directly abut each other in the fusion partner.

### Fusion Proteins

Also provided herein are heterologous fusion proteins that include: (i) a first amino acid sequence including a RNA-binding zinc finger domain (e.g., any of the exemplary RNA-binding zinc finger domains described herein) or an active fragment thereof; and (ii) a second amino acid sequence including a fusion partner (e.g., any of the exemplary fusion partners described herein).

In some embodiments of any of the fusion proteins described herein, the RNA-binding zinc finger domain can include an amino acid sequence that is at least 80% identical (e.g., at least 82% identical, at least 84% identical, at least 86% identical, at least 88% identical, at least 90% identical, at least 92% identical, at least 94% identical, at least 95% identical, at least 96% identical, at least 98% identical, at least 99% identical, or 100% identical) to SEQ ID NO: 1 or SEQ ID NO: 2.

In some embodiments of any of the fusion proteins described herein, the first sequence includes a first amino acid sequence of a first RNA-binding zinc finger domain (e.g., any of the exemplary RNA-binding zinc finger domains described herein) and a second amino acid sequence of a second RNA-binding zinc finger domain (e.g., any of the exemplary RNA-binding zinc finger domains described herein). For example, the first sequence encoding the RNA-binding zinc finger domain includes a first RNA-binding zinc finger domain including a sequence of SEQ ID NO: 1 or SEQ ID NO: 2 and a second RNA-binding zinc finger domain including a sequence of SEQ ID NO: 1 or SEQ ID NO: 2, or any combination thereof. In some embodiments where the fusion proteins include a first RNA-binding zinc finger domain (e.g., any of the exemplary RNA-binding zinc finger domains described herein) and a second RNA-binding zinc finger domain (e.g., any of the exemplary RNA-binding zinc finger domains described herein), the first RNA-binding zinc finger domain is directly adjacent to the second RNA-binding zinc finger domain. In some embodiments where fusion protein includes a first RNA-binding zinc finger domain (e.g., any of the exemplary RNA-binding zinc finger domains described herein) and a second RNA-binding zinc finger domain (e.g., any of the exemplary RNA-binding zinc finger domains described herein), a linker (e.g., any of the exemplary linkers described herein) is positioned between the first RNA-binding zinc finger domain and the second RNA-binding zinc finger domain. In some embodiments, the fusion protein includes three or more, four or more, five or more, or six or more RNA-binding zinc finger domains.

In some embodiments of any of the fusion proteins described herein, the fusion partner includes a RNA degrading enzyme (e.g., any of the exemplary RNA-degrading enzymes described herein (e.g., a PIN RNA endonuclease domain)). In some embodiments of any of the fusion proteins described herein, the fusion partner includes a reporter (e.g., any of the exemplary reporters described herein (e.g., a HA-tag)).

In some embodiments, the sequence of a fusion partner includes an amino acid sequence encoding a first fusion partner (e.g., any of the fusion partners described herein) and an amino acid sequence encoding a second fusion partner (e.g., any of the fusion partners described herein). In some embodiments where the amino acid sequence of the fusion partner includes a first fusion partner and a second fusion partner, the first fusion partner is a reporter sequence (e.g., any of the exemplary reporter sequences described herein or known in the art) and the second fusion partner is a RNA degrading enzyme (e.g., any of the RNA degrading enzymes described herein). For example, a fusion partner can include HA-Tag as first fusion partner and a PIN domain RNA endonuclease as a second fusion partner. In some embodiments, the amino acid sequence of a fusion partner includes a linker sequence (e.g., any of the exemplary linkers described herein) between the first fusion partner (e.g., any of the exemplary fusion partners described herein) and the second fusion partner (e.g., any of the exemplary fusion partners described herein). In some embodiments, the first fusion partner and the second fusion partner are directly adjacent to each other in the fusion protein.

In some embodiments of any of the fusion proteins described herein, the first amino acid sequence (e.g., any one or more of the exemplary RNA-binding zinc finger domains described herein) is positioned at the C-terminus of the second amino acid sequence (e.g., any one or more of the exemplary fusion partners described herein). In some embodiments of any of the fusion proteins described herein, the first amino acid sequence (e.g., any one or more of the exemplary RNA-binding zinc finger domains described herein) is positioned at the N-terminus of the second amino acid sequence (e.g., any one or more of the exemplary fusion partners described herein).

In some embodiments of any of the fusion proteins described herein, the fusion protein further includes a linker (e.g., any of the exemplary linkers described herein or known in the art) positioned between the first amino acid sequence (e.g., any one or more of the exemplary RNA-binding zinc finger domains described herein) and the second amino acid sequence (e.g., any one or more of the exemplary fusion partners described herein). In some examples, the linker includes a total of about 1 amino acid to about 50 amino acids or any of the subranges in between. In some examples, the linker includes (G₄S)ₙ (SEQ ID NO: 33), where n is 1, 2, 3, 4, or 5. In some embodiments, the linker can be a XTEN linker including a SEQ ID NO: 30-32. In some examples of any of the fusion proteins described herein, the first amino acid sequence is directly adjacent to the second amino acid sequence. In some embodiments of any of the fusion proteins described herein, the fusion protein further includes a secretion signal peptide that is operably linked to the first and/or second amino acid sequences.

In some embodiments, the fusion protein is encoded by a nucleic acid sequence that is at least 70% identical (e.g., at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical) to SEQ ID NOs: 11-23.

### Linker Sequences

In some embodiments, the linker sequence can be a flexible linker sequence. Non-limiting examples of linker sequences that can be used are described in Klein et al., Protein Engineering. Design & Selection 27(10):325-330, 2014; Priyanka et al., Protein Sci. 22(2):153-167, 2013. In some examples, the linker sequence is a synthetic linker sequence. In some embodiments, any of the fusion proteins described herein can include one, two, three, four, or five linker sequence(s) (e.g., the same or different linker sequences, e.g., any of the exemplary linker sequences described herein or known in the art).

In some embodiments, the linker sequence includes a total of about 1 amino acid to about 25 amino acids (e.g., about 1 amino acid to about 24 amino acids, about 1 amino acid to about 22 amino acids, about 1 amino acid to about 20 amino acids, about 1 amino acid to about 18 amino acids, about 1 amino acid to about 16 amino acids, about 1 amino acid to about 15 amino acids, about 1 amino acid to about 14 amino acids, about 1 amino acid to about 12 amino acids, about 1 amino acid to about 10 amino acids, about 1 amino acid to about 8 amino acids, about 1 amino acid to about 6 amino acids, about 1 amino acid to about 5 amino acids, about 1 amino acid to about 4 amino acids, about 1 amino acid to about 3 amino acids, about 1 amino acid to about 2 amino acids, about 2 amino acids to about 25 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 15 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 5 amino acids, about 2 amino acids to about 4 amino acids, about 2 amino acids to about 3 amino acids, about 4 amino acids to about 25 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 15 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 4 amino acids to about 5 amino acids, about 5 amino acids to about 25 amino acids, about 5 amino acids to about 24 amino acids, about 5 amino acids to about 22 amino acids, about 5 amino acids to about 20 amino acids, about 5 amino acids to about 18 amino acids, about 5 amino acids to about 16 amino acids, about 5 amino acids to about 15 amino acids, about 5 amino acids to about 14 amino acids, about 5 amino acids to about 12 amino acids, about 5 amino acids to about 10 amino acids, about 5 amino acids to about 8 amino acids, about 5 amino acids to about 6 amino acids, about 6 amino acids to about 25 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 15 amino acids, about 6 amino acids to about 14 amino acids, about 6 amino acids to about 12 amino acids, about 6 amino acids to about 10 amino acids, about 6 amino acids to about 8 amino acids, about 8 amino acids to about 25 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 15 amino acids, about 8 amino acids to about 14 amino acids, about 8 amino acids to about 12 amino acids, about 8 amino acids to about 10 amino acids, about 10 amino acids to about 25 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 15 amino acids, about 10 amino acids to about 14 amino acids, about 10 amino acids to about 12 amino acids, about 12 amino acids to about 25 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 15 amino acids, about 12 amino acids to about 14 amino acids, about 14 amino acids to about 25 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 14 amino acids to about 15 amino acids, about 15 amino acids to about 25 amino acids, about 15 amino acids to about 24 amino acids, about 15 amino acids to about 22 amino acids, about 15 amino acids to about 20 amino acids, about 15 amino acids to about 18 amino acids, about 15 amino acids to about 16 amino acids, about 16 amino acids to about 25 amino acids, about 16 amino acids to about 24 amino acids, about 16 amino acids to about 22 amino acids, about 16 amino acids to about 20 amino acids, about 16 amino acids to about 18 amino acids, about 18 amino acids to about 25 amino acids, about 18 amino acids to about 24 amino acids, about 18 amino acids to about 22 amino acids, about 18 amino acids to about 20 amino acids, about 20 amino acids to about 25 amino acids, about 20 amino acids to about 24 amino acids, about 20 amino acids to about 22 amino acids, about 22 amino acid to about 25 amino acids, about 22 amino acid to about 24 amino acids, or about 24 amino acid to about 25 amino acids).

In some embodiments, the linker sequence includes a total of about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 21 amino acids, about 22 amino acids, about 23 amino acids, about 24 amino acids, or about 25 amino acids in length.

In some embodiments, the linker sequence is rich in glycine (Gly or G) residues. In some embodiments, the linker sequence is rich in serine (Ser or S) residues. In some embodiments, the linker sequence is rich in glycine and serine residues. In some embodiments, the linker sequence has one or more glycine-serine residue pairs (GS), e.g., 1, 2, 3, 4, 5, 6, 7, 8, 9, or 10 or more GS pairs. In some embodiments, the linker sequence has one or more Gly-Gly-Gly-Gly-Ser (GGGGS) sequences, e.g., 1, 2, 3, 4, or 5 or more GGGGS (SEQ ID NO: 33) sequences.

In some embodiments, the linker can be a XTEN linker. Non-limiting examples of XTEN linkers include sequences of SEQ ID NO: 30-32.

### Gene Delivery Vectors

Also provided herein are gene delivery vectors that include any of the isolated nucleic acids described herein. In some embodiments, the gene delivery vectors are adeno-associated viral (AAV) vectors, lentiviral vectors, adenoviral vectors, or retroviral vectors. AAV vectors are generally described in, e.g., Asokan et al., Mol. Ther. 20: 699-708, 2012, and B. J. Carter, in “Handbook of Parvoviruses”, Ed., P. Tijsser, CRC Press, pp. 155-168, 1990. Adenoviral vectors are generally described in, e.g., Wold and Toth, Curr. Gene Ther. 13(6):421-433, 2013; Baron et al., Curr. Opin. Virol. 29:1-7, 2018; and Barry, Expert Rev. Vaccines 17(2): 163-173, 2018. Lentiviral vectors are generally described in, e.g., Milone and O'Doherty, Leukemia 32(7): 1529-1541, 2018, Zheng et al., Anat. Rec. 301(5): 825-836, 2018; and Cai et al., Curr. Gene Ther. 16(3): 194-206, 2016. Adenoviral vectors are generally described in, e.g., Tatsis et al., Mol. Ther. 10(4):616-629, 2004; Appaiahgari et al., Expert. Opin. Biol. Ther. 15(3):337-351, 2015; Coughlan, Methods Mol. Biol. 1108:23-40, 2014. Retroviral vectors are generally described in, e.g., Miller, Curr. Protoc. Hum. Genet. 80: Unit 12.5, 2014; Kim et al., Adv. Virus Res. 55:545-563, 2000; and Kurian et al., Mol. Pathol. 53(4):173-176, 2000.

Some embodiments of any of the gene delivery vectors described herein can include a promoter and/or enhancer (e.g., a tissue-specific promoter and/or enhancer, such as a brain-specific promoter and/or brain-specific enhancer) operably linked to a nucleic acid encoding a fusion protein, where the isolated nucleic acid sequence includes: (i) a first sequence encoding one or more RNA-binding zinc finger domains or an active fragment thereof (e.g., any of the exemplary RNA-binding zinc finger domains described herein); and (ii) a second sequence that encodes a fusion partner (e.g., any one or more of the exemplary fusion partners described herein).

In some embodiments, the gene delivery vector can be an AAV vector. For example, an AAV vector can be selected from the group of: an AAV2 vector, an AAV5 vector, and an AAV8 vector, an AAV1 vector, an AAV7 vector, an AAV9 vector, an AAV3 vector, an AAV6 vector, an AAV10 vector, and an AAV11 vector. In some embodiments, the gene delivery vector can be an AAV9 vector.

In some embodiments, the isolated nucleic acid includes a brain-specific promoter (e.g., any of the exemplary brain-specific promoters described herein) operably linked to the isolated nucleic acid sequence encoding a fusion protein (e.g., any of the exemplary fusion proteins described herein)). In some embodiments, the isolated nucleic acid includes a brain-specific enhancer operably linked to the isolated nucleic acid sequence encoding the fusion protein (e.g., any of the exemplary fusion proteins described herein). In some embodiments, the isolated nucleic acid includes a brain-specific enhancer and a brain-specific promoter operably linked to the isolated nucleic acid sequence encoding the fusion protein (e.g., any of the exemplary fusion proteins described herein).

In some embodiments, the gene delivery vectors described herein includes one or more (e.g., two, three, four, five, or six) of a promoter (e.g., any of the brain-specific promoters described herein or known in the art), an enhancer (e.g., any of the enhancers described herein or known in the art), a Kozak sequence (e.g., any of the Kozak sequences described herein or known in the art), an RNA splicing sequence, a polyadenylation (poly(A)) signal sequence (e.g., any of the poly(A) signals described herein), and an internal ribosome entry site (IRES) sequence (e.g., any of the IRES sequences described herein or known in the art).

In some embodiments, the gene delivery vector (e.g., AAV vector) include a total number of nucleotides of up to 5 kb. In some embodiments of any of the gene delivery vectors described herein, the gene delivery vector can include a total number of nucleotides in the range of about 500 to about 5,000 nucleotides, about 500 to about 4,500 nucleotides, about 500 to about 4,000 nucleotides, about 500 to about 3,500 nucleotides, about 500 to about 3,000 nucleotides, about 500 to about 2,500 nucleotides, about 500 to about 2,000 nucleotides, about 500 to about 1,500 nucleotides, about 500 to about 1,000 nucleotides, about 500 to about 800 nucleotides, about 600 to about 5,000 nucleotides, about 600 to about 4,500 nucleotides, about 600 to about 4,000 nucleotides, about 600 to about 3,500 nucleotides, about 600 to about 3,000 nucleotides, about 600 to about 2,500 nucleotides, about 600 to about 2,000 nucleotides, about 600 to about 1,500 nucleotides, about 600 to about 1,000 nucleotides, about 800 to about 5,000 nucleotides, about 800 to about 4,500 nucleotides, about 800 to about 4,000 nucleotides, about 800 to about 3,500 nucleotides, about 800 to about 3,000 nucleotides, about 800 to about 2,500 nucleotides, about 800 to about 2,000 nucleotides, about 800 to about 1,500 nucleotides, about 800 to about 1,000 nucleotides, about 1,000 to about 5,000 nucleotides, about 1,000 to about 4,500 nucleotides, about 1,000 to about 4,000 nucleotides, about 1,000 to about 3,500 nucleotides, about 1,000 to about 3,000 nucleotides, about 1,000 to about 2,500 nucleotides, about 1,000 to about 2,000 nucleotides, about 1,000 to about 1,500 nucleotides, about 1,500 to about 5,000 nucleotides, about 1,500 to about 4,500 nucleotides, about 1,500 to about 4,000 nucleotides, about 1,500 to about 3,500 nucleotides, about 1,500 to about 3,000 nucleotides, about 1,500 to about 2,500 nucleotides, about 1,500 to about 2,000 nucleotides, about 2,000 to about 5,000 nucleotides, about 2,000 to about 4,500 nucleotides, about 2,000 to about 4,000 nucleotides, about 2,000 to about 3,500 nucleotides, about 2,000 to about 3,000 nucleotides, about 2,000 to about 2,500 nucleotides, about 2,500 to about 5,000 nucleotides, about 2,500 to about 4,500 nucleotides, about 2,500 to about 4,000 nucleotides, about 2,500 to about 3,500 nucleotides, about 2,500 to about 3,000 nucleotides, about 3,000 to about 5,000 nucleotides, about 3,000 to about 4,500 nucleotides, about 3,000 to about 4,000 nucleotides, about 3,000 to about 3,500 nucleotides, about 3,500 to about 5,000 nucleotides, about 3,500 to about 4,500 nucleotides, about 3,500 to about 4,000 nucleotides, about 4,000 to about 5,000 nucleotides, about 4,000 to about 4,500 nucleotides, or about 4,500 to about 5,000 nucleotides.

### Methods of Introducing a Nucleic Acid in a Cell

Any of the isolated nucleic acids described herein can be introduced into any cell, e.g., a mammalian cell. Non-limiting examples of a mammalian cell include: a human cell, a rodent cell (e.g., a rat cell or a mouse cell), a rabbit cell, a dog cell, a cat cell, a porcine cell, or a non-human primate cell.

Methods of culturing cells are well known in the art. Cells can be maintained in vitro under conditions that favor cell proliferation, cell growth, and/or cell differentiation. For example, cells can be cultured by contacting a cell (e.g., any of the cells described herein) with a cell culture medium that includes supplemental growth factors to support cell viability and cell growth.

Methods of introducing nucleic acids (e.g., any of the exemplary nucleic acids described herein) and/or gene delivery vectors (e.g., any of the exemplary gene delivery vectors described herein (e.g., an AAV vector)) into cells (e.g., mammalian cells) are known in the art. Non-limiting examples of methods that can be used to introduce a nucleic acid (e.g., any of the exemplary nucleic acids described herein) and/or a gene delivery vector (e.g., any of the exemplary gene delivery vectors described herein (e.g., an AAV vector)) include: electroporation, lipofection, transfection, microinjection, calcium phosphate transfection, dendrimer-based transfection, anionic polymer transfection, cationic polymer transfection, transfection using highly branched organic compounds, cell-squeezing, sonoporation, optical transfection, magnetofection, particle-based transfection (e.g., nanoparticle transfection), transfection using liposomes (e.g., cationic liposomes), and viral transduction (e.g., lentiviral transduction, adenoviral transduction).

Also provided herein are methods of producing a fusion protein including: (a) culturing a cell (e.g., any of the cells described herein) including any of the isolated nucleic acids encoding any of the polypeptides described herein or any of the expression vectors described herein including a nucleic acid encoding any of the fusion proteins described herein in a culture medium under conditions sufficient to allow for the production of the fusion protein; and (b) harvesting the fusion protein from the host cell or the culture medium. In some embodiments of any of the methods described herein, the method further includes isolating the fusion protein encoded by any of the isolated nucleic acids described herein from cell culture medium or from a cell (e.g., any of the cells described herein) (e.g., through performance of one or more column chromatography steps, ultrafiltration/diafiltration, and/or viral inactivation). Non-limiting examples of methods of isolating a fusion protein encoded by any of the isolated nucleic acids described herein include: ion exchange chromatography (anionic or cation), metal-affinity chromatography, ligand-affinity chromatography, size exclusion chromatography, hydrophobic interaction chromatography, and precipitation (e.g., ammonium sulfate precipitation, polyethylene glycol precipitation).

In some embodiments of any of the methods described herein, the method further includes formulating the isolated fusion protein into a composition (e.g., a pharmaceutical composition).

Also provided herein are methods and compositions for specificity of transduction and/or infection, e.g., using any of the AAV capsid proteins or AAV virus serotypes. In some embodiments of any of the methods described herein, specificity of gene expression is determined, e.g., using any of the tissue-specific promoters and/or enhancers described herein.

### Promoters

In some embodiments, the gene delivery vector (e.g., any of the exemplary gene delivery vectors described herein) can include a promoter sequence. In some embodiments of any of the gene delivery vectors described herein, the promoter sequence is a brain-specific promoter. In some embodiments, a brain-specific promoter is a NSE, MAG, MBP, F4/80, GAP, vGLUT, GAD a GFAP or SYN-1 promoter (see, e.g., Ingusci et al., Front. Pharmacol., 10:724 (2019)). In some embodiments, the promoter is an H1 promoter. In some embodiments, a promoter is a ubiquitous promoter. Non-limiting examples of ubiquitous promoters include CAG, EF1α, UBC, SV40, CMV, or PGK (see, e.g., Ingusci et al., Front. Pharmacol., 10:724 (2019)).

### Enhancers

In some embodiments, the gene delivery vector (e.g., any of the exemplary gene delivery vectors described herein) can include an enhancer sequence. In some embodiments of any of the gene delivery vectors described herein, the enhancer sequence is a brain-specific enhancer (e.g., Hb9) (see, e.g., Lukashchuk et al., Met. Clin. Dev., 3: doi:10.1038/mtm.2015.55 (2016)). In some embodiments, an enhancer sequence is a CMV enhancer, a CAG enhancer, or a cHS4 enhancer ((see, e.g., Ingusci et al., Front. Pharmacol., 10:724 (2019)).

### Poly(A) Signal

In some embodiments, the gene delivery vector (e.g., any of the exemplary gene delivery vectors described herein) can include a polyadenylation (poly(A)) signal sequence. Poly(A) tails are added to most nascent eukaryotic messenger RNAs (mRNAs) at their 3′ end during a complex process that includes cleavage of the primary transcript and a coupled polyadenylation reaction driven by the poly(A) signal sequence. In some embodiments of any of the gene delivery vectors described herein, the gene delivery vector can include a poly(A) signal sequence at the 3′ end of the isolated nucleic acid encoding a fusion protein (e.g., any of the fusion proteins described herein).

The term “polyadenylation” refers to the covalent linkage of a polyadenylyl moiety, or its modified variant, to the 3′ end of a mRNA molecule. A poly(A) tail is a long sequence of adenine nucleotides (e.g., 40, 50, 100, 200, 500, 1000) added to the pre-mRNA by a polyadenylate polymerase.

The term “poly(A) signal sequence” or “poly(A) signal” is a sequence that triggers the endonuclease cleavage of a mRNA and the addition of a sequence of adenosine to the 3′end of the cleaved mRNA. Non-limiting examples of poly(A) signals include: bovine growth hormone (bGH) poly(A) signal, human growth hormone (hGH) poly(A) signal. In some embodiments of any of the AAV vectors described herein, the AAV vector can include a poly(A) signal sequence that includes the sequence AATAAA or variations thereof. Additional examples of poly(A) signal sequences are known in the art.

### Internal Ribosome Entry Site (IRES) and 2A-Self-Cleaving Peptide

In some embodiments, the gene delivery vector (e.g., any of the exemplary gene delivery vectors described herein) can include an internal ribosome entry site (IRES) sequence. An IRES sequence is used to produce more than one polypeptide from a single gene transcript, and forms a complex secondary structure that allows translation initiation to occur from any position with an mRNA immediately downstream from where the IRES is located. See, e.g., Pelletier and Sonnenberg, Mol. Cell. Biol. 8(3): 1103-1112, 1988; and Hellen et al., Genes Dev. 15(13): 1593-1612, 2001. Non-limiting examples of IRES sequences include those from, e.g., hepatitis C virus (HCV), poliovirus (PV), hepatitis A virus (HAV), foot and mouth disease virus (FMDV).

In some embodiments, the gene delivery vector (e.g., any of the exemplary gene delivery vectors described herein) can include a sequence encoding a “self-cleaving” 2A peptide (e.g., T2A, P2A, E2A, or F2A). A self-cleaving 2A-peptide is used to produce more than one polypeptide from a single gene transcript by inducing ribosomal skipping during translation.

In some embodiments, the nucleic acid sequences are operably linked to a promoter or are operably linked to other nucleic acid sequences using a self-cleaving 2A peptide or an IRES sequence.

### Compositions and Kits

Also provided herein are compositions (e.g., pharmaceutical compositions) that include any of the gene delivery vectors (e.g., AAV vectors) described herein or any of the heterologous fusion proteins described herein. Any of the pharmaceutical compositions can include any of the gene delivery vectors described herein and one or more (e.g., 1, 2, 3, 4, or 5) pharmaceutically or physiologically acceptable carriers, diluents, or excipients. In some embodiments, any of the pharmaceutical compositions described herein can include one or more buffers (e.g., a neutral-buffered saline, a phosphate-buffered saline (PBS)), one or more carbohydrates (e.g., glucose, mannose, sucrose, dextran, or mannitol), one or more proteins, polypeptides, or amino acids (e.g., glycine), one or more antioxidants, one or more chelating agents (e.g., glutathione or EDTA), one or more preservatives, and/or a pharmaceutically acceptable carrier (e.g., PBS, saline, or bacteriostatic water).

In some embodiments, any of the pharmaceutical compositions described herein can further include one or more (e.g., 1, 2, 3, 4, or 5) agents that promote the entry of any of the gene delivery vectors described herein into a cell (e.g., a mammalian cell) (e.g., a liposome or cationic lipid).

In some embodiments, any of the gene delivery vectors described herein can be formulated using natural and/or synthetic polymers. Non-limiting examples of polymers that can be included in any of the pharmaceutical compositions described herein can include, but are not limited to: poloxamer, chitosan, dendrimers and poly(lactic-co-glycolic acid) (PLGA) polymers.

In some embodiments of any of the pharmaceutical compositions described herein, a single dose of a pharmaceutical composition can include a total sum amount of at least 1 ng (e.g., at least 2 ng, at least 4 ng, at least 5 ng, at least 6 ng, at least 8 ng, at least 10 ng, at least 15 ng, at least 20 ng, at least 30 ng, at least 40 ng, at least 50 ng, at least 60 ng, at least 80 ng, at least 100 ng, at least 120 ng, at least 200 ng, at least 400 ng, at least 500 ng, at least 1 μg, at least 2 μg, at least 4 μg, at least 6 μg, at least 8 μg, at least 10 μg, at least 12 μg, at least 14 μg, at least 16 μg, at least 18 μg, at least 20 μg, at least 24 μg, at least 25 μg, at least 30 μg, at least 40 μg, at least 50 μg, at least 60 μg, at least 80 μg, at least 100 μg, at least 120 μg, at least 140 μg, at least 150 μg, at least 160 μg, at least 180 μg, or at least 200 μg) of any of the gene delivery vectors described herein or any of the fusion proteins described herein, e.g., in a buffered solution.

In some embodiments of any of the pharmaceutical compositions described herein, a single dose of a pharmaceutical composition can include a total sum amount of at least 10⁸ vg/kg, at least 10⁹ vg/kg, at least 10¹⁰ vg/kg, at least 10¹¹ vg/kg, at least 10¹² vg/kg, or at least 10¹³ vg/kg) of any of the gene delivery vectors described herein, e.g., in a buffered solution.

In some embodiments of any of the pharmaceutical compositions described herein, a single dose of a pharmaceutical composition can include a total sum amount of at least 1×10⁸ vg/kg, at least 1×10⁹ vg/kg, at least 1×10¹⁰ vg/kg, at least 1×10¹¹ vg/kg, at least 1×10¹² vg/kg, or at least 1×10¹³ vg/kg) of any of the gene delivery vectors described herein, e.g., in a buffered solution. In some embodiments of any of the pharmaceutical compositions described herein, a single dose of a pharmaceutical composition can include a total sum amount of about 1×10⁸ vg/kg to about 1×10¹⁵ vg/kg, about 5×10⁸ vg/kg to about 5×10¹⁴ vg/kg, about 1×10⁹ vg/kg to about 1×10¹⁴ vg/kg, about 5×10⁹ vg/kg to about 5×10¹³ vg/kg, about 1×10¹⁰ vg/kg to about 1×10¹³ vg/kg, or about 5×10¹⁰ vg/kg to about 5×10¹² vg/kg in a buffered solution.

The pharmaceutical compositions provided herein can be, e.g., formulated to be compatible with their intended route of administration. In some embodiments, the compositions are formulated for subcutaneous, intramuscular, intravenous, or intrahepatic administration. In some examples, the compositions include a therapeutically effective amount of any of the gene delivery vectors described herein.

Also provided are kits that include any of the compositions (e.g., pharmaceutical compositions), isolated nucleic acids, gene delivery vectors, or fusion proteins described herein. In some embodiments, a kit can include a solid composition (e.g., a lyophilized composition including any of the gene delivery vectors described herein) and a liquid for solubilizing the lyophilized composition.

In some embodiments, a kit can include a pre-loaded syringe including any of the pharmaceutical compositions described herein.

In some embodiments, the kit includes a vial including any of the pharmaceutical compositions described herein (e.g., formulated as an aqueous pharmaceutical composition).

In some embodiments, the kit can include instructions for performing any of the methods described herein.

### Cells

Also provided herein is a mammalian cell (e.g., a peripheral mammalian cell, a mammalian neural cell, e.g., a human neural cell) that includes any of the gene delivery vectors, fusion proteins, or isolated nucleic acids described herein. Also provided is a mammalian cell (e.g., a mammalian neural cell, e.g. a human neural cell) that is transduced with any of the gene delivery vectors described herein, edited using lentiviral or CRISPR technologies, or otherwise engineered or modified to express any of the fusion proteins described herein. Skilled practitioners will appreciate that the gene delivery vectors described herein can be introduced into any mammalian cell (e.g., any neural cell), that a variety of technologies can be utilized for modifying the genome of mammalian cells, and that such modified human cells that secrete fusion proteins can be utilized as cell therapies. Non-limiting examples of gene delivery vectors and methods for introducing gene delivery vectors into mammalian cells (e.g., any neural cell, e.g., a human neural cell) are described herein.

In some embodiments, the mammalian cell is a human cell, a rodent cell (e.g., a rat cell or a mouse cell), a rabbit cell, a dog cell, a cat cell, a porcine cell, or a non-human primate cell. In some embodiments, the mammalian cell is present in a subject (e.g., a human subject). In some embodiments, the mammalian cell is an autologous cell obtained from a subject (e.g., a human subject) and cultured ex vivo. In some embodiments, the mammalian cell is in vitro.

### Methods

Also provided herein are methods decreasing a level of RNA having a G₄C₂ hexanucleotide expanded repeat in the central nervous system (CNS) of a subject in need thereof that include administering to the subject any of the gene delivery vectors described herein, any of the fusion proteins described herein, or any of the pharmaceutical compositions described herein.

Also provided herein are methods for treating a subject having a disease associated with G₄C₂ hexanucleotide expanded repeats administering to the subject a therapeutically effective amount of any of the gene delivery vectors described herein, any of the fusion proteins described herein, or any of the pharmaceutical compositions described herein. Additional non-limiting aspects of the gene delivery vectors, fusion proteins, pharmaceutical compositions, kits, and methods are described herein and can be used in any combination without limitation.

In some embodiments of these methods, the method can result in at least a 2.0-fold (e.g., at least a 2.5-fold, at least a 3.0-fold, at least a 3.5-fold, at least a 4.0-fold, at least a 4.5-fold, at least a 5.0-fold, at least a 6.0-fold, at least a 7.0-fold, at least a 8.0-fold, at least a 9.0-fold, at least a 10-fold, at least a 15-fold, at least a 20-fold, at least a 30-fold, at least a 40-fold, at least a 50-fold, at least a 60-fold, at least a 80-fold, at least a 100-fold, at least a 120-fold, or at least a 150-fold) decrease in the level of RNA having a G₄C₂ hexanucleotide repeat in the CNS of a subject, e.g., as compared to the level of RNA having a G₄C₂ hexanucleotide repeat in the CNS of the subject prior to the administering. In some examples of these methods, the method can result from about a 2-fold to about a 150-fold, about a 2-fold to about a 100-fold, about a 2-fold to about a 50-fold, about a 2-fold to about a 25-fold, about a 2-fold to about a 10-fold, about a 2-fold to about a 5-fold, about a 5-fold to about a 150-fold, about a 5-fold to about a 100-fold, about a 5-fold to about a 50-fold, about a 5-fold to about a 25-fold, about a 5-fold to about a 10-fold, about a 10-fold to about a 150-fold, a 10-fold to about a 100-fold, about a 10-fold to about a 50-fold, about a 10-fold to about a 25-fold, about a 25-fold to about a 150-fold, about a 25-fold to about a 100-fold, or about a 25-fold to about a 50-fold, decrease in the level of RNA having a G₄C₂ hexanucleotide repeat in the CNS of a subject, e.g., as compared to the level of RNA having a G₄C₂ hexanucleotide repeat in the CNS of the subject prior to the administering.

In some embodiments, the level of RNA having a G₄C₂ hexanucleotide repeat in the CNS of a subject can be detected using imaging (e.g., using an antibody conjugated with a detectable agent, e.g., fluorophore or a chemiluminescent molecule, e.g., using CT, MRI, CAT scan, or ultrasound). In some embodiments, the level of RNA having a G₄C₂ hexanucleotide repeat in the CNS of a subject can be determined by detecting a level of RNA having a G₄C₂ hexanucleotide repeat in cerebrospinal fluid obtained from the subject (e.g., using immunoprecipitation, Western blotting, immunohistochemistry, immunofluorescence, enzyme-linked immunosorbent assay (ELISA), or proteomics). In some embodiments, the level of RNA having a G₄C₂ hexanucleotide repeat in the CNS of a subject can be assessed indirectly by detecting an improvement in one or more symptoms of a CNS disease or disorder (e.g., a G₄C₂ hexanucleotide repeat-associated disease) in a subject (e.g., a decrease in the duration, severity, number, or frequency of one or more symptoms of the CNS disease or disorder (e.g., a G₄C₂ hexanucleotide repeat-associated disease in a subject), e.g., as assessed by a medical professional (e.g., a physician). Non-limiting examples of G₄C₂ hexanucleotide repeat-associated disease include frontal temporal dementia (TFD) and amyotrophic lateral sclerosis (ALS).

In some embodiments of any of the methods described herein, the gene delivery vector can be a plasmid, an artificial chromosome, or a viral vector (e.g., an adenoviral vector, a lentivirus vector, a retroviral vector, or an AAV vector (e.g., any of the AAV vectors described herein).

In some embodiments of any of the methods described herein, the gene delivery vector can be formulated for intravenous administration, intrahepatic administration, subcutaneous administration, or intramuscular administration.

In some embodiments of any of the methods described herein, the subject is cat, a dog, a goat, a human, a non-human primate, a rodent (e.g., a mouse or a rat), a pig, or a sheep. In some embodiments of any of the methods described herein, the subject is human and is an adult, juvenile, a teenager, a child, a toddler, an infant, or a newborn.

In some embodiments of any of the methods described herein, the subject has or is at risk of developing a CNS disorder or disease (e.g., a G₄C₂ hexanucleotide repeat-associated disease). In some embodiments of any of the methods described herein, the subject has been previously identified or diagnosed as having a CNS disorder or disease (e.g., a G₄C₂ hexanucleotide repeat-associated disease).

Also provided herein are methods decreasing a level of RNA having a C₄G₂ hexanucleotide expanded repeat in the central nervous system (CNS) of a subject in need thereof that include administering to the subject any of the gene delivery vectors described herein, any of the fusion proteins described herein, or any of the pharmaceutical compositions described herein.

Also provided herein are methods for treating a subject having a disease associated with C₄G₂ hexanucleotide expanded repeats administering to the subject a therapeutically effective amount of any of the gene delivery vectors described herein, any of the fusion proteins described herein, or any of the pharmaceutical compositions described herein. Additional non-limiting aspects of the gene delivery vectors, fusion proteins, pharmaceutical compositions, kits, and methods are described herein and can be used in any combination without limitation.

In some embodiments of these methods, the method can result in at least a 2.0-fold (e.g., at least a 2.5-fold, at least a 3.0-fold, at least a 3.5-fold, at least a 4.0-fold, at least a 4.5-fold, at least a 5.0-fold, at least a 6.0-fold, at least a 7.0-fold, at least a 8.0-fold, at least a 9.0-fold, at least a 10-fold, at least a 15-fold, at least a 20-fold, at least a 30-fold, at least a 40-fold, at least a 50-fold, at least a 60-fold, at least a 80-fold, at least a 100-fold, at least a 120-fold, or at least a 150-fold) decrease in the level of RNA having a C₄G₂ hexanucleotide repeat in the CNS of a subject, e.g., as compared to the level of RNA having a C₄G₂ hexanucleotide repeat in the CNS of the subject prior to the administering. In some examples of these methods, the method can result from about a 2-fold to about a 150-fold, about a 2-fold to about a 100-fold, about a 2-fold to about a 50-fold, about a 2-fold to about a 25-fold, about a 2-fold to about a 10-fold, about a 2-fold to about a 5-fold, about a 5-fold to about a 150-fold, about a 5-fold to about a 100-fold, about a 5-fold to about a 50-fold, about a 5-fold to about a 25-fold, about a 5-fold to about a 10-fold, about a 10-fold to about a 150-fold, a 10-fold to about a 100-fold, about a 10-fold to about a 50-fold, about a 10-fold to about a 25-fold, about a 25-fold to about a 150-fold, about a 25-fold to about a 100-fold, or about a 25-fold to about a 50-fold, decrease in the level of RNA having a C₄G₂ hexanucleotide repeat in the CNS of a subject, e.g., as compared to the level of RNA having a C₄G₂ hexanucleotide repeat in the CNS of the subject prior to the administering.

In some embodiments, the level of RNA having a C₄G₂ hexanucleotide repeat in the CNS of a subject can be detected using imaging (e.g., using an antibody conjugated with a detectable agent, e.g., fluorophore or a chemiluminescent molecule, e.g., using CT, MRI, CAT scan, or ultrasound). In some embodiments, the level of RNA having a C₄G₂ hexanucleotide repeat in the CNS of a subject can be determined by detecting a level of RNA having a G₄C₂ hexanucleotide repeat in cerebrospinal fluid obtained from the subject (e.g., using immunoprecipitation, Western blotting, immunohistochemistry, immunofluorescence, enzyme-linked immunosorbent assay (ELISA), or proteomics). In some embodiments, the level of RNA having a G₄C₂ hexanucleotide repeat in the CNS of a subject can be assessed indirectly by detecting an improvement in one or more symptoms of a CNS disease or disorder (e.g., a C₄G₂ hexanucleotide repeat-associated disease) in a subject (e.g., a decrease in the duration, severity, number, or frequency of one or more symptoms of the CNS disease or disorder (e.g., a C₄G₂ hexanucleotide repeat-associated disease in a subject), e.g., as assessed by a medical professional (e.g., a physician). Non-limiting examples of C₄G₂ hexanucleotide repeat-associated disease include frontal temporal dementia (TFD) and amyotrophic lateral sclerosis (ALS).

In some embodiments of any of the methods described herein, the subject has or is at risk of developing a CNS disorder or disease (e.g., a C₄G₂ hexanucleotide repeat-associated disease). In some embodiments of any of the methods described herein, the subject has been previously identified or diagnosed as having a CNS disorder or disease (e.g., a C₄G₂ hexanucleotide repeat-associated disease).

Also provided herein are methods of decreasing one or both of (i) a level of RNA having a C₄G₂ hexanucleotide repeat (CNS) and (ii) a level of RNA having a G₄C₂ hexanucleotide repeat in the central nervous system of a subject in need thereof, including administering to the subject any of the gene delivery vectors described herein, any of the heterologous fusion proteins described herein, or any of the pharmaceutical compositions described herein. In some embodiments, the administering results in at least a 2-fold reduction in one or both of (i) the level of RNA having a C₄G₂ hexanucleotide repeat and (ii) the level of RNA having a G₄C₂ hexanucleotide repeat in the CNS of the subject as compared to the level of RNA having (i) a C₄G₂ hexanucleotide repeat and (ii) a G₄C₂ hexanucleotide repeat in the CNS of the subject prior to administering.

In another aspect, this document features a method of treating a subject having one or both of (i) a C₄G₂ hexanucleotide repeat-associated disease or disorder and (ii) a G₄C₂ hexanucleotide repeat-associated disease or disorder including administering to the subject a therapeutically effective amount of any of the gene delivery vectors described herein, any of the heterologous fusion proteins described herein, or any of the pharmaceutical compositions described herein. In some embodiments, the subject is previously diagnosed or identified as having one or both of (i) a C₄G₂ hexanucleotide repeat-associated disease or disorder and (ii) a G₄C₂ hexanucleotide repeat-associated disease or disorder. In some embodiments, the C₄G₂ hexanucleotide repeat-associated disease or disorder is frontotemporal dementia (FTD) or amyotrophic lateral sclerosis (ALS). In some embodiments, the G₄C₂ hexanucleotide repeat-associated disease or disorder is frontotemporal dementia (FTD) or amyotrophic lateral sclerosis (ALS). In some embodiments, the administering results in at least a 2-fold reduction in the level of RNA having one or both of (i) a C₄G₂ hexanucleotide repeat and (ii) a G₄C₂ hexanucleotide repeat in the CNS of the subject as compared to the level of RNA having one or both of (i) a C₄G₂ hexanucleotide repeat and (ii) a G₄C₂ hexanucleotide repeat in the CNS of the subject prior to administering.

Method Tracking and/or Measuring RNA

Also provided herein are methods of tracking a RNA having a G₄C₂ hexanucleotide repeat or measuring the amount of a RNA having a G₄C₂ hexanucleotide repeat in a cell where the includes administering to a cell any of the isolated nucleic acid sequences described herein or any the gene delivery vehicle described herein. In some embodiments, the fusion protein includes a reporter where the fusion protein binds to RNA having the G₄C₂ hexanucleotide repeat in the cell and the reporter is used to determine the location of the RNA having the G₄C₂ hexanucleotide repeat and/or determining the amount of RNA having the G₄C₂ hexanucleotide repeat based on detection of the reporter.

Also provided herein are methods of tracking a RNA having a C₄G₂ hexanucleotide repeat or measuring the amount of a RNA having a C₄G₂ hexanucleotide repeat in a cell where the includes administering to a cell any of the isolated nucleic acid sequences described herein or any the gene delivery vehicle described herein. In some embodiments, the fusion protein includes a reporter where the fusion protein binds to RNA having the C₄G₂ hexanucleotide repeat in the cell and the reporter is used to determine the location of the RNA having the C₄G₂ hexanucleotide repeat and/or determining the amount of RNA having the C₄G₂ hexanucleotide repeat based on detection of the reporter.

Without further description, it is believed that one of ordinary skill in the art can, using the preceding description and the following illustrative examples, make and utilize the gene delivery vectors of the present disclosure and practice the claimed methods. The following working examples specifically point out various aspects of the present disclosure, and are not to be construed as limiting in any way the remainder of the disclosure.

### EXAMPLES

### Example 1. Construction of Plasmids and Production of AAV-RNA-Binding Zinc Finger Domain Vectors

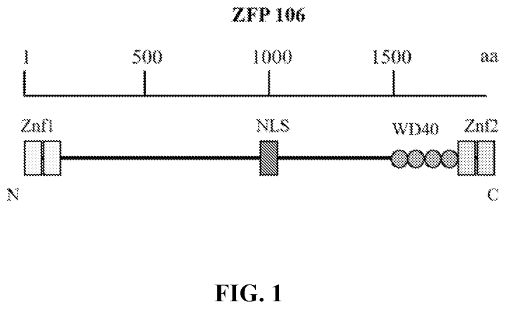

A plasmid was constructed with a nucleic acid sequence encoding: Znf1, a RNA-binding zinc finger domain (SEQ ID NO: 1), fused to a PIN domain. FIG. 1 shows the basic protein structure of human ZFP106. ZFP106 contains two C2H2 zinc finger domains (Znf1 and Znf2), a nuclear localization signal (NLS) and WD40 repeat domains.

Expression of the nucleic acid sequence was linked to a brain specific promoter. The transgene (Znf1) was then packaged in AAV vectors serotyped with AAV9 capsid. Alternatively Znf2, or Znf1 and Znf2, were each fused to a PIN domain packed in AAV vectors serotyped with AAV9 capsid. Exemplary AAV vector inserts include SEQ ID NO; 23-29. AAV vector inserts for each of the Znf1-PIN, Znf2-PIN, or Znf1+2-PIN were cloned into an AAV vector and used for transfection and or production of AAV.

### Example 2. In Vitro Targeting of G₄C₂ and C₄G₂ RNA with Zinc Finger Fusion Proteins

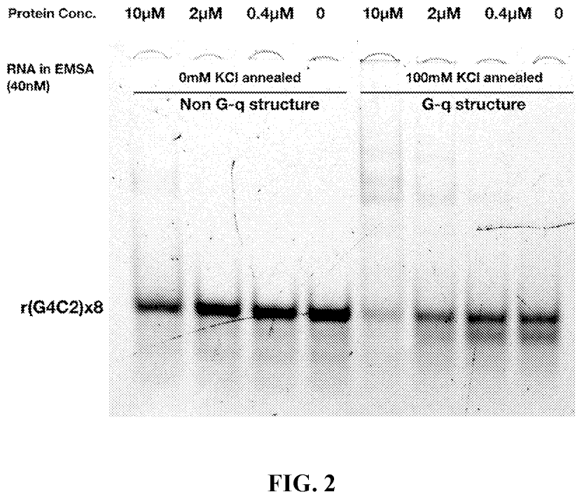

An RNA electrophoretic mobility-shift assay (EMSA) was used to confirm binding of purified Znf1 (“Znf1” used interchangeably with “Z1”) that binds directly to (GGGGCC)₈ in vitro (FIG. 2). As shown in FIG. 2, purified Znf1 binds to both G-q and non-G-q structures (G₄C₂)₈ as measured using EMSA

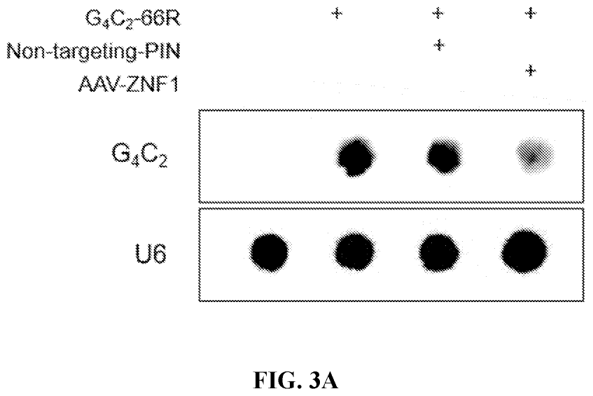

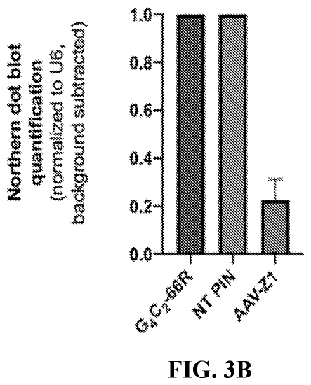

In a set of experiments, Znf1, Znf2, or Znf1+Znf2 (Znf1+2) were fused to a PIN domain and cloned into an AAV vector. COS-M6 cells were transfected with (G₄C₂)₆₆ RNA, G₄C₂-targeting ZFP AAVs (AAVZnf1, AAV-Znf2, or AAV-Znf1+2) or a non-targeting AAV construct expressing GFP (AAV-GFP). FIG. 3A shows a RNA dot blot of (G₄C₂)₆₆ RNA levels following treatment with the various conditions. Quantification of the RNA dot blot shows reduced (G₄C₂)₆₆ RNA in each construct tested including AAV-Znf1, AAV-Znf2, AAV-Znf1+2 as compared to the negative control ((G₄C₂)₆₆ RNA only) and a non-targeting AAV construct (non-targeting PIN) (FIG. 3B).

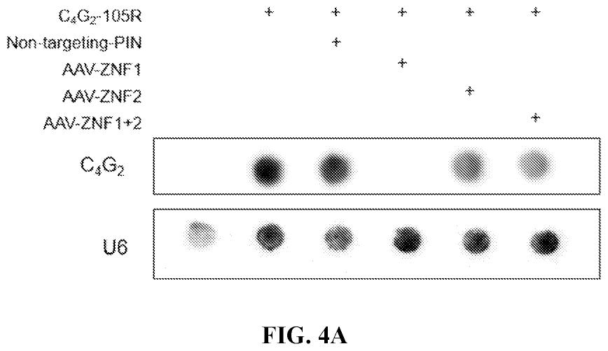

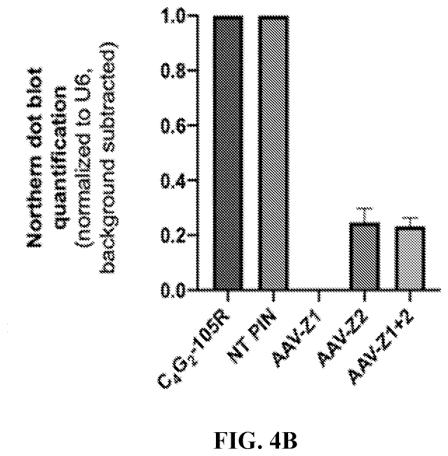

In a set of experiments, Znf1, Znf2 or Znf1+Znf2 (Znf1+2) were fused to a PIN domain and cloned into an AAV vector. COS-M6 cells were transfected with (C₄G₂)₁₀₅ RNA, C₄G₂-targeting ZFP AAVs (AAV-Znf1, AAV-Znf2, AAV-Znf1+2) or a non-targeting AAV construct (non-targeting PIN). U6 snRNA served as a loading control. FIG. 4A shows a RNA dot blot of C₄G₂ RNA levels following treatment with the various conditions. Quantification of the RNA dot blot shows reduced (C₄G₂)₁₀₅ RNA in each construct testing including AAV-Znf1, AAV-Znf2, AAV-Znf1+2 as compared to the negative control ((C₄G₂)₁₀₅ RNA only) and non-targeting AAV construct (non-targeting PIN) (FIG. 43).

### Example 3. AAV-Z1 Construct does not Target all GC-Rich Transcripts In Vitro

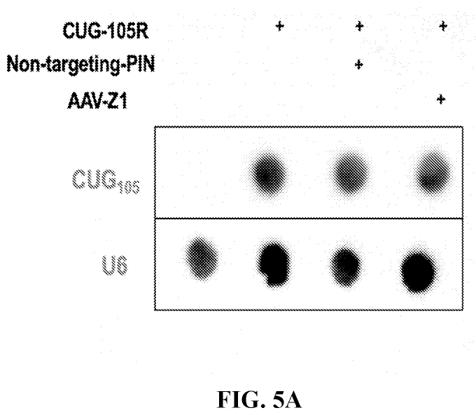

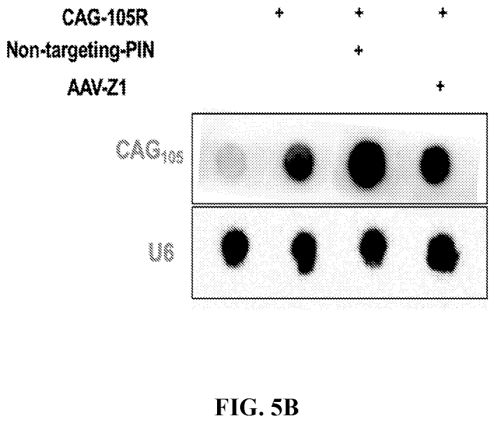

As shown in FIG. 5A, RNA dot blot of (CUG) expression levels in COS-M6 cells transfected with (CUG)₁₀₅, G₄C₂+C₂G₄-targeting ZFP AAVs (AAV-Znf1) or a non-targeting AAV construct (non-targeting PIN) showed no reduction in (CUG)₁₀₅ levels as compared to controls. U6 snRNA served as a loading control. As shown in FIG. 5B, RNA dot blot of (CAG) expression levels in COS-M6 cells transfected with (CAG)₁₀₅ and a G₄C₂+C₂G₄-targeting AAV-Znf1 or a non-targeting AAV construct (non-targeting PIN) showed no reduction in (CAG)₁₀₅ levels as compared to controls. U6 snRNA served as a loading control.

### Example 4. Administration of a scAAV9-Z1 to a Human Patient to Treat C9-ALS

A human patient is identified as being in need of treatment with a composition containing AAV9-Z1 to decrease the level of RNA having a G₄C₂ hexanucleotide repeat in the central nervous system (CNS). A cerebrospinal (CSF) sample is collected from the patient and used to measure the level of RNA having a G₄C₂ hexanucleotide repeat. G₄C₂ hexanucleotide repeats are detected using repeat-primed RT-PCR coupled with fluorescent fragment analysis (see e.g., He et al., Neurol. Genet., 2: DOI 10.1212/NXG.0000000000000071 (2016)). When G₄C₂ RNA levels are present above a given threshold, the patient is identified has having elevated levels of RNA having a G₄C₂ repeat.

An AAV9 gene delivery vector including a sequence that encodes for the Z1-PIN (“Z1”) fusion protein is selected for treatment based, at least in part, on the levels of RNA having G₄C₂ repeats. A dosage of 1×10¹⁰ vg/kg of pharmaceutical composition containing AAV9-Z1 is administered via intracranial injection to the identified patient. At a follow-up appointment at 3-months, 6-months, 9 months, and 1 year, the patient's level of RNA having a G₄C₂ hexanucleotide repeat is measured using repeat-primed RT-PCR coupled with fluorescent fragment analysis as described in Example 4. Based on the patient's level of RNA having a G₄C₂ hexanucleotide repeat, zero, one, or more additional intracranial injections are administered to the patient. In at least some cases, the administration of the pharmaceutical composition results in at least a 2-fold reduction in the level of RNA having a G₄C₂ hexanucleotide repeat in the CNS of the subject as compared to the level of RNA having a G₄C₂ hexanucleotide repeat in the CNS of the subject prior to administering the pharmaceutical composition.

### Example 5. Viral Delivery of scAAV9-Z1 to C9-ALS Patient-Specific Spinal Organoids

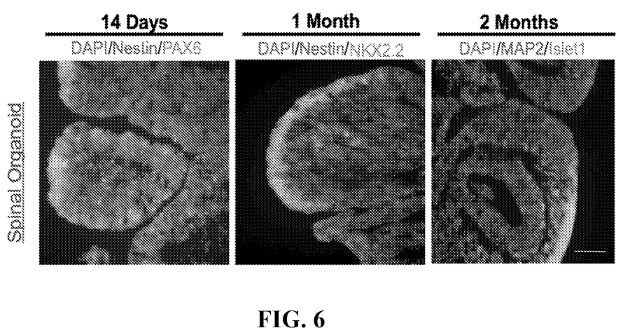

In a set of experiments, AAV-Z1 (Z1-PIN fusion protein) was tested in an in vitro disease model of C9-ALS using spinal organoids derived from induced pluripotent stem cells (iPSCs) from C9-CLS patients. Specifically, ventral spinal organoids were generated from C9-ALS patient iPSCs and transduced with AAV-Z1 to determine Z1's ability to knockdown G₄C₂/C₄G₂-mediated RNA foci in patient-specific cells most impacted by disease. The cellular composition of the spinal organoids, include interneuron & oligodendrocyte (NKX2.2) and mature motor neurons (Islet1+ cells) recapitulating the cytoarchitecture of the human spinal cord (FIG. 6). Neural progenitor (PAX6⁺, Nestin⁺ cells) are observed within the developing spinal organoids as early as 14 days post-differentiation. Over time, neural progenitors start to differentiate into interneuron and oligodendrocyte precursors (NKX2.2⁺) and eventually into mature motor neurons (Islet1⁺ cells).

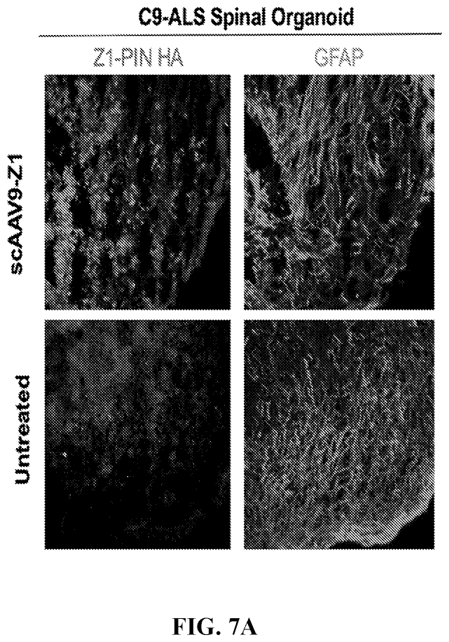

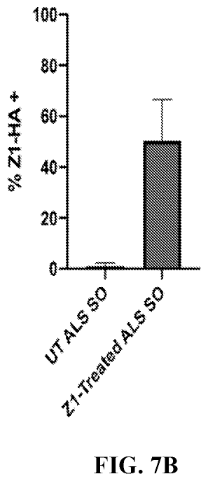

Both C9-ALS and control ventral spinal organoids were transduced with scAAV9-Z1 or an scAAV9 with a non-targeting PIN starting at 30 days post-differentiation. Organoids were cultured for an additional 30 days and isolated at day 60. Bio-distribution of Z1 was detected and quantified via immunohistochemistry with an anti-HA antibody directed to the HA-tag in the scAAV9-Z1 construct. FIG. 7A shows staining for HA-tagged Z1-PIN and GFAP. Quantification showed greater than 40% of cells positive for Z1 expression (FIG. 7B).

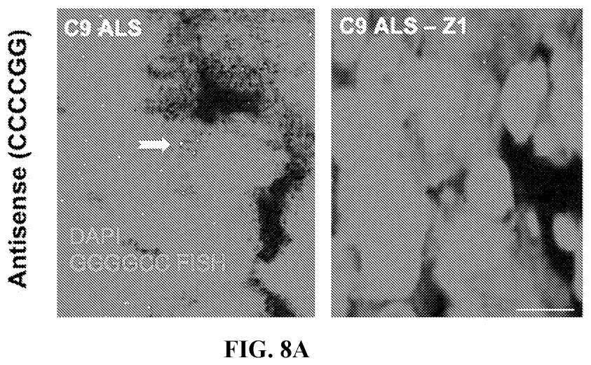

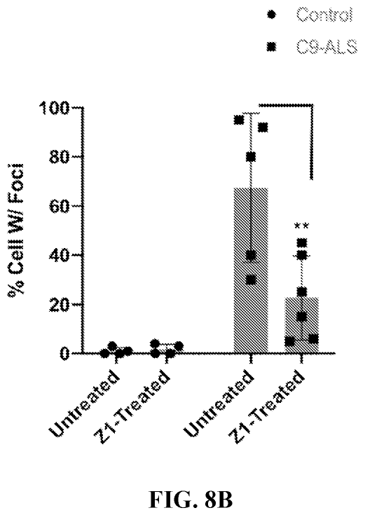

Detection and quantification of both sense and antisense RNA foci were detected using fluorescent in situ hybridization (FISH). FIG. 8A shows representative images of FISH with a sense FISH probe to the antisense C₄G₂ repeat within C9-ALS patient iPSC-derived spinal organoids transduced with or without scAAV9-Z1. White arrow points to at least one GGGGCC FISH probe hybridized to at least one C₄G₂ repeat (see FIG. 8A). FIG. 8B shows quantification of the percentage of cells with antisense C₄G₂ foci in C9-ALS spinal organoids transduced with or without scAAV9-Z1 as compared to non-disease spinal organoids transduced with and without scAAV9-Z1. FIGS. 8A-8B show a reduction in antisense C₄G₂ RNA foci in scAAV9-Z1 treated spinal organoids as compared to untreated and non-disease controls.

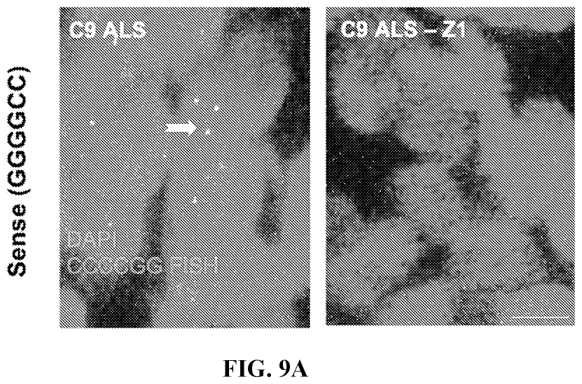

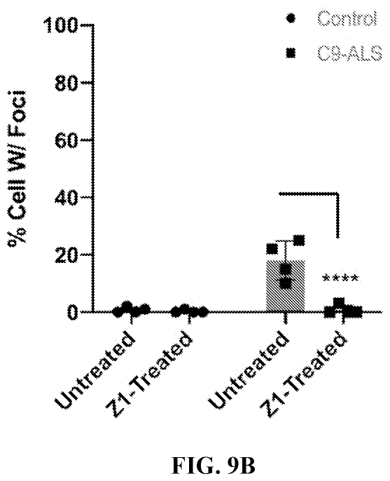

FIG. 9A shows representative images of FISH with an antisense FISH probe to the sense G₄C₂ repeat within C9-ALS patient iPSC-derived spinal organoids transduced with or without scAAV9-Z1. White arrow points to at least one CCCCGG FISH probe hybridized to at least one G₄C₂ repeat (see FIG. 9A). FIG. 9B shows quantification of the percentage of cells with sense RNA foci in C9-ALS spinal organoids transduced with or without scAAV9-Z1 as compared to non-disease spinal organoids transduced with and without scAAV9-Z1. FIGS. 9A-9B show a reduction in sense G₄C₂ RNA foci in scAAV9-Z1 treated spinal organoids as compared to untreated and non-disease controls.

Data was analyzed using a one-way ANOVA followed by Tukey's HSD posthoc comparisons. *=p<0.05, **=p<0.01, ***=p<0.001, ****=p<0.0001. Scale bars, 25 μm

### EMBODIMENTS

Embodiment A1. An isolated nucleic acid encoding a fusion protein, wherein the isolated nucleic acid comprises:

(i) a first sequence encoding a RNA-binding zinc finger domain or a fragment thereof comprising:

an amino acid sequence of
(SEQ ID NO: 1)
HECRVCGVTEVGLSAYAKHISGQLH,
or
an amino acid sequence of
(SEQ ID NO: 2)
YRCWWHGCSLIFGVVDHLKQHLLTDH;

and

(ii) a second sequence encoding a fusion partner.

Embodiment A2. The isolated nucleic acid of embodiment A1, wherein the RNA-binding zinc finger domain comprises an amino acid sequence that is at least 90% identical to SEQ ID NO: 1.

Embodiment A3. The isolated nucleic acid of embodiment A1, wherein the RNA-binding zinc finger domain comprises an amino acid sequence that is at least 95% identical to SEQ ID NO: 1.

Embodiment A4. The isolated nucleic acid of embodiment A1, wherein the RNA-binding zinc finger domain comprises an amino sequence of SEQ ID NO: 1.

Embodiment A5. The isolated nucleic acid of embodiment A1, wherein the RNA-binding zinc finger domain comprises an amino acid sequence that is at least 90% identical to SEQ ID NO: 2.

Embodiment A6. The isolated nucleic acid of embodiment A1, wherein the RNA-binding zinc finger domain comprises an amino acid sequence that is at least 95% identical to SEQ ID NO: 2.

Embodiment A7. The isolated nucleic acid of embodiment A1, wherein the RNA-binding zinc finger domain comprises an amino acid sequence of SEQ ID NO: 2.

Embodiment A8. The isolated nucleic acid of any one of embodiments A1-A7, wherein the first sequence further comprises an amino acid sequence encoding a second RNA-binding zinc finger domain.

Embodiment A9. The isolated nucleic acid of embodiment A8, wherein the first RNA-binding zinc finger domain and the second RNA-binding zinc finger domain are identical.

Embodiment A10. The isolated nucleic acid of embodiment A8, wherein the first RNA-binding zinc finger domain and the second RNA-binding zinc finger domain are different.

Embodiment A11. The isolated nucleic acid of embodiment A8, wherein the first RNA-binding zinc finger domain comprises SEQ ID NO: 1, and wherein the second RNA-binding zinc finger domain comprises SEQ ID NO: 2.

Embodiment A12. The isolated nucleic acid of embodiment A8, wherein the first RNA-binding zinc finger domain comprises SEQ ID NO: 2, and wherein the second RNA-binding zinc finger domain comprises SEQ ID NO: 1.

Embodiment A13. The isolated nucleic acid of embodiment A8, wherein the first RNA-binding zinc finger domain comprises SEQ ID NO: 1, and wherein the second RNA-binding zinc finger domain comprises SEQ ID NO: 1.

Embodiment A14. The isolated nucleic acid of embodiment A8, wherein the first RNA-binding zinc finger domain comprises SEQ ID NO: 2, and wherein the second RNA-binding zinc finger domain comprises SEQ ID NO: 2.

Embodiment A15. The isolated nucleic acid of any one of embodiments A8-A14, wherein the first RNA-binding zinc finger domain is directly adjacent to the second RNA-binding zinc finger domain.

Embodiment A16. The isolated nucleic acid of any one of embodiments A8-A14, wherein the isolated nucleic acid further comprises a sequence encoding a linker positioned between the first RNA-binding zinc finger domain and the second RNA-binding zinc finger domain.

Embodiment A17. The isolated nucleic acid of embodiment A20, wherein the linker comprises about 1 amino acids to about 20 amino acids.

Embodiment A18. The isolated nucleic acid of embodiment A20, wherein the linker between the first RNA-binding zinc finger domain and the second RNA-binding zinc finger domain comprises (G4S)ₙ, wherein n is 1, 2, 3, 4, or 5.

Embodiment A19. The isolated nucleic acid of any one of embodiments A8-A18, wherein the first RNA-binding zinc finger domain is 5′ positioned relative to the second RNA-binding zinc finger domain.

Embodiment A20. The isolated nucleic acid of any one of embodiments A8-A18, wherein the second RNA-binding zinc finger domain is 5′ positioned relative to the first RNA-binding zinc finger domain.

Embodiment A21. The isolated nucleic acid of any one of embodiments A1-A20, wherein the first sequence encoding the RNA-binding zinc finger domain comprises three or more RNA-binding zinc finger domains.

Embodiment A22. The isolated nucleic acid of any one of embodiments A1-A20, wherein the first sequence is directly adjacent to the second sequence.

Embodiment A23. The isolated nucleic acid of any one of embodiments A1-A20, wherein the isolated nucleic acid further comprises a sequence encoding a linker positioned between the first sequence and the second sequence.

Embodiment A24. The isolated nucleic acid of embodiment A23, wherein the linker comprises a total of about 1 amino acid to about 20 amino acids.

Embodiment A25. The isolated nucleic acid of embodiment A23, wherein the linker comprises (G₄S)ₙ, wherein n is 1, 2, 3, 4, or 5.

Embodiment A26. The isolated nucleic acid of embodiment A23, wherein the linker is XTEN (SEQ ID NO: 30).

Embodiment A27. The isolated nucleic acid of any one of embodiments A1-A26, wherein the fusion partner comprises a reporter or a RNA degrading enzyme.

Embodiment A28. The isolated nucleic acid of embodiment A27, wherein the fusion partner comprises a sequence encoding a reporter.

Embodiment A29. The isolated nucleic acid of embodiment A28, wherein the reporter is selected from the group consisting of a HIS-tag, Flag™ tag, a HA-Tag, a fluorescent protein, a luminescent protein, and a detectable label.

Embodiment A30. The isolated nucleic acid of embodiment A27, wherein the fusion partner comprises a sequence encoding a RNA degrading enzyme comprising an endonucleases, a 5′ exonucleases, or a 3′ exonucleases.

Embodiment A31. The isolated nucleic acid of embodiment A30, wherein the fusion partner comprises a sequence encoding a human endonuclease, wherein the endonuclease cleaves single stranded RNA.

Embodiment A32. The isolated nucleic acid of embodiment A31, wherein the endonuclease comprises a PIN (PilT N-terminal domain) RNA endonuclease domain or active fragment thereof.

Embodiment A33. The isolated nucleic acid of any one of embodiments A1-A32, wherein the second sequence further comprises a sequence encoding a second fusion partner.

Embodiment A34. The isolated nucleic acid of embodiment A33, wherein the first fusion partner is directly adjacent to the second fusion partner.

Embodiment A35. The isolated nucleic acid of embodiment A33, wherein the isolated nucleic acid further comprises a sequence encoding a linker positioned between the first fusion partner and the second fusion partner.

Embodiment A36. The isolated nucleic acid of embodiment A35, wherein the linker comprises a total of about 1 amino acid to about 20 amino acids.

Embodiment A37. The isolated nucleic acid of embodiment A35, wherein the linker comprises (G₄S)n, wherein n is 1, 2, 3, 4, or 5.

Embodiment A38. The isolated nucleic acid of embodiment A35, wherein the linker is SEQ ID NO: 30.

Embodiment A39. The isolated nucleic acid of any one of embodiments A33-A38, wherein the first fusion partner is a reporter sequence, and wherein the second fusion partner is a RNA degrading enzyme.

Embodiment A40. The isolated nucleic acid of any one of embodiments A33-A39, wherein the first fusion partner is 5′ positioned relative to the second fusion partner.

Embodiment A41. The isolated nucleic acid of any one of embodiments A33-A39, wherein the second fusion partner is 5′ positioned to the first fusion partner.

Embodiment A42. The isolated nucleic acid of any one of embodiments A1-A41, wherein the first sequence is 5′ positioned relative to the second sequence.

Embodiment A43. The isolated nucleic acid of any one of embodiments A1-A41, wherein the second sequence is 5′ positioned relative to the first sequence.

Embodiment A44. The isolated nucleic acid of any one of embodiments A1-A43, wherein the isolated nucleic acid further comprises a promoter operably linked to the first and second sequence.

Embodiment A45. The isolated nucleic acid of embodiment A44, wherein the promoter is a tissue-specific promoter.

Embodiment A46. A gene delivery vector comprising the isolated nucleic acid sequence of any one of embodiments A1-A45.

Embodiment A47. The gene delivery vector of embodiment A46, wherein the gene delivery vector is selected from the group consisting of an adenoviral vector, an adeno associated viral (AAV) vector, a lentiviral vector, and a retroviral vector.

Embodiment A48. The gene delivery vector of embodiment A47, wherein the gene delivery vector is an AAV vector.

Embodiment A49. The gene delivery vector of embodiment A48, wherein the AAV vector is an AAV9 vector.

Embodiment A50. A pharmaceutical composition comprising the gene delivery vector of any one of embodiments A46-A49.

Embodiment A51. A kit comprising the pharmaceutical composition of embodiment A50.

Embodiment A52. A mammalian cell transfected with the isolated nucleic acid of any one of embodiments 1-45 or transduced with the gene delivery vector of any one of embodiments 46-49.

Embodiment A53. The mammalian cell of embodiment A52, wherein the mammalian cell is transfected or transduced in vitro.

Embodiment A54. The mammalian cell of embodiment A52, wherein the mammalian cell is transduced in vivo.

Embodiment A55. A method of decreasing a level of RNA having a G₄C₂ hexanucleotide repeat in the central nervous system (CNS) of a subject in need thereof, comprising administering to the subject a gene delivery vector of any one of embodiments A46-A49 or a pharmaceutical composition of embodiment A50.

Embodiment A56. The method of embodiment A55, wherein the administering results in at least a 2-fold reduction in the level of RNA having a G₄C₂ hexanucleotide repeat in the CNS of the subject as compared to the level of RNA having a G₄C₂ hexanucleotide repeat in the CNS of the subject prior to administering.

Embodiment A57. The method of embodiment A55, wherein administering the gene delivery vector or pharmaceutical composition comprises intravenous injection, intravenous infusion, intracranial injection, or extracranial injection.

Embodiment A58. A method of treating a subject having a G₄C₂ hexanucleotide repeat-associated disease or disorder comprising administering to the subject a therapeutically effective amount of a gene delivery vector of any one of embodiments A46-A49 or a pharmaceutical composition of embodiment A50.

Embodiment A59. The method of embodiment A58, wherein the subject is previously diagnosed or identified as having a G₄C₂ hexanucleotide repeat-associated disease or disorder.

Embodiment A60. The method of embodiment A59, wherein the G₄C₂ hexanucleotide repeat-associated disease or disorder is frontotemporal dementia (FTD) or amyotrophic lateral sclerosis (ALS).

Embodiment A61. The method of embodiment A58, wherein administering the gene delivery vector or pharmaceutical composition comprises intravenous injection, intravenous infusion, intracranial injection, or extracranial injection.

Embodiment A62. The method of embodiment A58, wherein the administering results in at least a 2-fold reduction in the level of RNA having a G₄C₂ hexanucleotide repeat in the CNS of the subject as compared to the level of RNA having a G₄C₂ hexanucleotide repeat in the CNS of the subject prior to administering.

Embodiment A63. A method of tracking a RNA having a G₄C₂ hexanucleotide repeat or measuring the amount of a RNA having a G₄C₂ hexanucleotide repeat in a cell, the method comprising

administering to a cell the isolated nucleic acid of embodiments A1-A45 or the gene delivery vehicle of any one of embodiments A46-A49, wherein the fusion protein comprises a reporter, and wherein the fusion protein binds to RNA having the G₄C₂ hexanucleotide repeat in the cell, and

determining the location of the RNA having the G₄C₂ hexanucleotide repeat and/or determining the amount of RNA having the G₄C₂ hexanucleotide repeat based on detection of the reporter.

Embodiment A64. The method of 63, wherein the location and/or amount of the RNA having the G₄C₂ hexanucleotide repeat in the cell is determined using fluorescence microscopy or an equivalent thereof.

Embodiment A65. The method of 64, wherein the cell is derived from a tissue, a biopsy, a serum sample, or a blood sample.

Embodiment A66. A heterologous fusion protein comprising:

(i) a first amino acid sequence comprising a RNA-binding zinc finger domain or a fragment thereof comprising:
a sequence of HECRVCGVTEVGLSAYAKHISGQLH (SEQ ID NO: 1), or
a sequence of YRCWWHGCSLIFGVVDHLKQHLLTDH (SEQ ID NO: 2); and

(ii) a second amino acid sequence comprising a fusion partner.

Embodiment A67. The fusion protein of embodiment A66, wherein the RNA-binding zinc finger domain comprises an amino acid sequence that is at least 90% identical to SEQ ID NO: 1.

Embodiment A68. The fusion protein of embodiment A66, wherein the RNA-binding zinc finger domain comprises an amino acid sequence that is at least 95% identical to SEQ ID NO: 1.

Embodiment A69. The fusion protein of embodiment A66, wherein the RNA-binding zinc finger domain comprises an amino sequence of SEQ ID NO: 1.

Embodiment A70. The fusion protein of embodiment A66, wherein the RNA-binding zinc finger domain comprises an amino acid sequence that is at least 90% identical to SEQ ID NO: 2.

Embodiment A71. The fusion protein of embodiment A66, wherein the RNA-binding zinc finger domain comprises an amino acid sequence that is at least 95% identical to SEQ ID NO: 2.

Embodiment A72. The fusion protein of embodiment A66, wherein the RNA-binding zinc finger domain comprises an amino acid sequence of SEQ ID NO: 2.

Embodiment A73. The fusion protein of any one of embodiments A66-A73, wherein the first amino acid sequence further comprises an amino acid sequence of a second RNA-binding zinc finger domain.

Embodiment A74. The fusion protein of embodiment A73, wherein the first RNA-binding zinc finger domain and the second RNA-binding zinc finger domain are identical.

Embodiment A75. The fusion protein of embodiment A73, wherein the first RNA-binding zinc finger domain and the second RNA-binding zinc finger domain are different.

Embodiment A76. The fusion protein of embodiment A73, wherein the first RNA-binding zinc finger domain comprises SEQ ID NO: 1, and wherein the second RNA-binding zinc finger domain comprises SEQ ID NO: 2.

Embodiment A77. The fusion protein of embodiment A73, wherein the first RNA-binding zinc finger domain comprises SEQ ID NO: 2, and wherein the second RNA-binding zinc finger domain comprises SEQ ID NO: 1.

Embodiment A78. The fusion protein of embodiment A73, wherein the first RNA-binding zinc finger domain comprises SEQ ID NO: 1, and wherein the second RNA-binding zinc finger domain comprises SEQ ID NO: 1.

Embodiment A79. The fusion protein of embodiment A73, wherein the first RNA-binding zinc finger domain comprises SEQ ID NO: 2, and wherein the second RNA-binding zinc finger domain comprises SEQ ID NO: 2.

Embodiment A80. The fusion protein of any one of embodiments A73-A79, wherein the first RNA-binding zinc finger domain is directly adjacent to the second RNA-binding zinc finger domain.

Embodiment A81. The fusion protein of any one of embodiments A73-A79, wherein the fusion protein further comprises a linker positioned between the first RNA-binding zinc finger domain and the second RNA-binding zinc finger domain.

Embodiment A82. The fusion protein of embodiment A81, wherein the linker comprises about 1 amino acids to about 20 amino acids.

Embodiment A83. The fusion protein of embodiment A81, wherein the linker between the first RNA-binding zinc finger domain and the second RNA-binding zinc finger domain comprises (G₄S)n, wherein n is 1, 2, 3, 4, or 5.

Embodiment A84. The fusion protein of any one of embodiments A73-A83, wherein the first RNA-binding zinc finger domain is positioned at the C-terminally of the second RNA-binding zinc finger domain.

Embodiment A85. The fusion protein of any one of embodiments A73-A83, wherein the second RNA-binding zinc finger domain is N-terminally positioned relative to the first RNA-binding zinc finger domain.

Embodiment A86. The fusion protein of any one of embodiments A66-A85, wherein the second amino acid sequence comprises three or more RNA-binding zinc finger domains.

Embodiment A87. The fusion protein of any one of embodiments A66-A85, wherein the first amino acid sequence is directly adjacent to the second amino acid sequence.

Embodiment A88. The fusion protein of any one of embodiments A66-A85, wherein the fusion protein further comprises a linker positioned between the first amino acid sequence and the second amino acid sequence.

Embodiment A89. The fusion protein of embodiment A88, wherein the linker comprises a total of about 1 amino acid to about 20 amino acids.

Embodiment A90. The fusion protein of embodiment A88, wherein the linker comprises (G₄S)n, wherein n is 1, 2, 3, 4, or 5.

Embodiment A91. The fusion protein of embodiment A88, wherein the linker is XTEN (SEQ ID NO: 30).

Embodiment A92. The fusion protein of any one of embodiments A66-A88, wherein the fusion partner comprises a reporter or a RNA degrading enzyme.

Embodiment A93. The fusion protein of embodiment A92, wherein the fusion partner comprises an amino acid sequence of a reporter.

Embodiment A94. The fusion protein of embodiment A93, wherein the reporter is selected from the group consisting of a HIS-tag, Flag™ tag, a HA-Tag, a fluorescent protein, a luminescent protein, and a detectable label.

Embodiment A95. The fusion protein of embodiment A92, wherein the fusion partner comprises a RNA degrading enzyme comprising endonucleases, 5′ exonucleases, or 3′ exonucleases.

Embodiment A96. The fusion protein of embodiment A95, wherein the fusion partner comprises a human endonuclease, wherein the endonuclease cleaves single stranded RNA.

Embodiment A97. The fusion protein of embodiment A96, wherein the endonuclease comprises a PIN RNA endonuclease domain or active fragment thereof.

Embodiment A98. The fusion protein of any one of embodiments A66-A97, wherein the second amino acid sequence further comprises a second fusion partner.

Embodiment A99. The fusion protein of embodiment A98, wherein the first fusion partner is directly adjacent to the second fusion partner.

Embodiment A100. The fusion protein of embodiment A98, wherein the fusion protein further comprises a linker positioned between the first fusion partner and the second fusion partner.

Embodiment A101. The fusion protein of embodiment A100, wherein the linker comprises a total of about 1 amino acid to about 20 amino acids.

Embodiment A102. The fusion protein of embodiment A100, wherein the linker comprises (G₄S)n, wherein n is 1, 2, 3, 4, or 5.

Embodiment A103. The fusion protein of embodiment A100, wherein the linker comprises SEQ ID NO: 30.

Embodiment A104. The fusion protein of any one of embodiments A98-A103, wherein the first fusion partner is a reporter sequence, and wherein the second fusion partner is a RNA degrading enzyme.

Embodiment A105. The fusion protein of any one of embodiments A98-A104, wherein the first fusion partner is N-terminally positioned relative to the second fusion partner.

Embodiment A106. The fusion protein of any one of embodiments A98-A104, wherein the second fusion partner is N-terminally positioned to the first fusion partner.

Embodiment A107. The fusion protein of any one of embodiments A66-A106, wherein the first amino acid sequence is N-terminally positioned relative to the second amino acid sequence.

Embodiment A108. The fusion protein of any one of embodiments A66-A106, wherein the second sequence is N-terminally positioned relative to the first sequence.

Embodiment A109. A pharmaceutical composition comprising the fusion protein of any one of embodiments A66-A108.

Embodiment A110. A kit comprising the pharmaceutical composition of embodiment A109.

Embodiment A111. A method of decreasing a level of RNA having a G₄C₂ hexanucleotide repeat in the central nervous system (CNS) of a subject in need thereof, comprising administering to the subject a heterologous fusion protein of any one of embodiments A66-A106 or a pharmaceutical composition of embodiment A109.

Embodiment A112. The method of embodiment A111, wherein the administering results in at least a 2-fold reduction in the level of RNA having a G₄C₂ hexanucleotide repeat in the CNS of the subject as compared to the level of RNA having a G₄C₂ hexanucleotide repeat of the subject prior to administering.

Embodiment A1413. The method of embodiment A111, wherein administering the heterologous fusion protein or the pharmaceutical composition comprises intravenous injection, intravenous infusion, intracranial injection, or extracranial injection.

Embodiment A114. A method of treating a subject having a G₄C₂ hexanucleotide repeat-associated disease or disorder comprising administering to the subject a therapeutically effective amount of a heterologous fusion protein of any one of embodiments A66-A106 or a pharmaceutical composition of embodiment A109.

Embodiment A115. The method of embodiment A114, wherein the subject is previously diagnosed or identified as having a G₄C₂ hexanucleotide repeat-associated disease or disorder.

Embodiment A116. The method of embodiment A115, wherein the G₄C₂ hexanucleotide repeat-associated disease or disorder is frontotemporal dementia (FTD) or amyotrophic lateral sclerosis (ALS).

Embodiment A117. The method of embodiment A114, wherein administering the heterologous fusion protein or pharmaceutical composition comprises intravenous injection, intravenous infusion, intracranial injection, or extracranial injection.

Embodiment A118. The method of embodiment A114, wherein the administering results in at least a 2-fold reduction in the level of RNA having a G₄C₂ hexanucleotide repeat in the CNS of the subject as compared to the level of RNA having a G₄C₂ hexanucleotide repeat of the subject prior to administering.

Embodiment A119. A method of tracking an RNA having a G₄C₂ hexanucleotide repeat or measuring the amount of an RNA having a G₄C₂ hexanucleotide repeat in a cell, the method comprising

administering to a cell the heterologous fusion protein of any one of embodiments A66-A106, wherein the fusion protein comprises a reporter, and wherein the fusion protein binds to RNA having the G₄C₂ hexanucleotide repeat in the cell, and

determining the location of the RNA having the G₄C₂ hexanucleotide repeat and/or determining the amount of RNA having the G₄C₂ hexanucleotide repeat based on detection of the reporter.

Embodiment A120. The method of embodiment A119, wherein the location and/or amount of the RNA having the G₄C₂ hexanucleotide repeat in the cell is determined using fluorescence microscopy or an equivalent thereof.

Embodiment A121. The method of embodiment A120, wherein the cell is derived from a tissue, a biopsy, a serum sample, or a blood sample.

Embodiment A122. A method of decreasing a level of RNA having a C₄G₂ hexanucleotide repeat in the central nervous system (CNS) of a subject in need thereof, comprising administering to the subject a gene delivery vector of any one of embodiments A46-A49 or a pharmaceutical composition of embodiment A50.

Embodiment A123. The method of embodiment A122, wherein the administering results in at least a 2-fold reduction in the level of RNA having a C₄G₂ hexanucleotide repeat in the CNS of the subject as compared to the level of RNA having a C₄G₂ hexanucleotide repeat in the CNS of the subject prior to administering.

Embodiment A124. The method of embodiment 122, wherein administering the gene delivery vector or pharmaceutical composition comprises intravenous injection, intravenous infusion, intracranial injection, or extracranial injection.

Embodiment A125. A method of treating a subject having a C₄G₂ hexanucleotide repeat-associated disease or disorder comprising administering to the subject a therapeutically effective amount of a gene delivery vector of any one of embodiments A46-A49 or a pharmaceutical composition of embodiment A50.

Embodiment A126. The method of embodiment A125, wherein the subject is previously diagnosed or identified as having a C₄G₂ hexanucleotide repeat-associated disease or disorder.

Embodiment A127. The method of embodiment A126, wherein the C₄G₂ hexanucleotide repeat-associated disease or disorder is frontotemporal dementia (FTD) or amyotrophic lateral sclerosis (ALS).

Embodiment A128. The method of embodiment A125, wherein administering the gene delivery vector or pharmaceutical composition comprises intravenous injection, intravenous infusion, intracranial injection, or extracranial injection.

Embodiment A129. The method of embodiment A125, wherein the administering results in at least a 2-fold reduction in the level of RNA having a C₄G₂ hexanucleotide repeat in the CNS of the subject as compared to the level of RNA having a C₄G₂ hexanucleotide repeat in the CNS of the subject prior to administering.

Embodiment A130. A method of tracking a RNA having a C₄G₂ hexanucleotide repeat or measuring the amount of a RNA having a C₄G₂ hexanucleotide repeat in a cell, the method comprising

administering to a cell the isolated nucleic acid of embodiments A1-A45 or the gene delivery vehicle of any one of embodiments A46-A49, wherein the fusion protein comprises a reporter, and wherein the fusion protein binds to RNA having the C₄G₂ hexanucleotide repeat in the cell, and

determining the location of the RNA having the G₄C₂ hexanucleotide repeat and/or determining the amount of RNA having the C₄G₂ hexanucleotide repeat based on detection of the reporter.

Embodiment A131. The method of embodiment A130, wherein the location and/or amount of the RNA having the C₄G₂ hexanucleotide repeat in the cell is determined using fluorescence microscopy or an equivalent thereof.

Embodiment A132. The method of embodiment A131, wherein the cell is derived from a tissue, a biopsy, a serum sample, or a blood sample.

Embodiment A133. A method of decreasing a level of RNA having a C₄G₂ hexanucleotide repeat in the central nervous system (CNS) of a subject in need thereof, comprising administering to the subject a heterologous fusion protein of any one of embodiments A66-A106 or a pharmaceutical composition of embodiment A109.

Embodiment A134. The method of embodiment A133, wherein the administering results in at least a 2-fold reduction in the level of RNA having a C₄G₂ hexanucleotide repeat in the CNS of the subject as compared to the level of RNA having a C₄G₂ hexanucleotide repeat of the subject prior to administering.

Embodiment A135. The method of embodiment A133, wherein administering the heterologous fusion protein or the pharmaceutical composition comprises intravenous injection, intravenous infusion, intracranial injection, or extracranial injection.

Embodiment A136. A method of treating a subject having a C₄G₂ hexanucleotide repeat-associated disease or disorder comprising administering to the subject a therapeutically effective amount of a heterologous fusion protein of any one of embodiments A66-A106 or a pharmaceutical composition of embodiment A109.

Embodiment A137. The method of embodiment A136, wherein the subject is previously diagnosed or identified as having a C₄G₂ hexanucleotide repeat-associated disease or disorder.

Embodiment A138. The method of embodiment A137, wherein the C₄G₂ hexanucleotide repeat-associated disease or disorder is frontotemporal dementia (FTD) or amyotrophic lateral sclerosis (ALS).

Embodiment A139. The method of embodiment A136, wherein administering the heterologous fusion protein or pharmaceutical composition comprises intravenous injection, intravenous infusion, intracranial injection, or extracranial injection.

Embodiment A140. The method of embodiment A136, wherein the administering results in at least a 2-fold reduction in the level of RNA having a C₄G₂ hexanucleotide repeat in the CNS of the subject as compared to the level of RNA having a C₄G₂ hexanucleotide repeat of the subject prior to administering.

Embodiment A141. A method of tracking a RNA having a C₄G₂ hexanucleotide repeat or measuring the amount of a RNA having a C₄G₂ hexanucleotide repeat in a cell, the method comprising

administering the heterologous fusion protein of any one of embodiments A66-A106, wherein the fusion protein comprises a reporter, and wherein the fusion protein binds to RNA having the C₄G₂ hexanucleotide repeat in the cell, and

determining the location of the RNA having the G₄C₂ hexanucleotide repeat and/or determining the amount of RNA having the C₄G₂ hexanucleotide repeat based on detection of the reporter.

Embodiment A142. The method of embodiment A141, wherein the location and/or amount of the RNA having the C₄G₂ hexanucleotide repeat in the cell is determined using fluorescence microscopy or an equivalent thereof.

Embodiment A143. The method of embodiment A142, wherein the cell is derived from a tissue, a biopsy, a serum sample, or a blood sample.

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

Sequence Appendix

| SEQ | | |
|---|---|---|
| ID | | |
| NO: | Molecule | Sequence |
| 1 | RNA- | HECRVCGVTEVGLSAYAKHISGQLH |
| | binding zinc | |
| | finger | |
| | domain 1 | |
| 2 | RNA- | YRCWWHGCSLIFGVVDHLKQHLLTDH |
| | binding zinc | |
| | finger | |
| | domain 2 | |
| 3 | Nucleic | catgaatgccgcgtgtgcggcgtgaccgaagtgggcctgagcgcgtatgcgaaacatattagcggcc |
| | acid | agctgcat |
| | sequence | |
| | encoding | |
| | RNA- | |
| | binding | |
| | zinc finger | |
| | domain 1 | |
| 4 | Nucleic | tatcgctgctggtggcatggctgcagcctgatttttggcgtggtggatcatctgaaacagcatctgctgac |
| | acid | cgatcat |
| | sequence | |
| | encoding | |
| | RNA- | |
| | binding | |
| | zinc finger | |
| | domain 2 | |
| 5 | ZNF1_HA_ | catgaatgccgcgtgtgcggcgtgaccgaagtgggcctgagcgcgtatgcgaaacatattagcggccagctgc |
| | 3′ | attacccatacgatgttccagattacgct |
| 6 | ZNF2_HA_ | tatcgctgctggtggcatggctgcagcctgatttttggcgtggtggatcatctgaaacagcatctgctgaccgatcat |
| | 3′ | tacccatacgatgttccagattacgct |
| 7 | HA_ZNF1_ | tacccatacgatgttccagattacgctcatgaatgccgcgtgtgcggcgtgaccgaagtgggcctgagcgcgtat |
| | 5′ | gcgaaacatattagcggccagctgcat |
| 8 | ZNF2_HA_ | tatcgctgctggtggcatggctgcagcctgatttttggcgtggtggatcatctgaaacagcatctgctgaccgatcat |
| | 5′ | acccatacgatgttccagattacgct |
| 9 | ZNF1&2HA_ | catgaatgccgcgtgtgcggcgtgaccgaagtgggcctgagcgcgtatgcgaaacatattagcggccagctgc |
| | 3′ | atggcagcggaagtgggcatgaatgccgcgtgtgcggcgtgaccgaagtgggcctgagcgcgtatgcgaaac |
| | | atattagcggccagctgcattacccatacgatgttccagattacgct |
| 10 | ZNF1&2- | catgaatgccgcgtgtgcggcgtgaccgaagtgggcctgagcgcgtatgcgaaacatattagcggccagctgc |
| | HA_5′ | atggcagcggaagtgggcatgaatgccgcgtgtgcggcgtgaccgaagtgggcctgagcgcgtatgcgaaac |
| | | atattagcggccagctgcatacccatacgatgttccagattacgct |
| 11 | ZNF1_HA_ | atgcagatggagctcgaaatcaggccgctgttcctcgtgccggacactaatggttttatagatcacttggcgtcctt |
| | XTEN_PIN_ | ggctagacttctggaaagccgaaagtatatattggtagtgccgttgattgtaattaacgaattggatgggttggcga |
| | 5′ | aaggacaagagactgatcacagagcaggaggctacgcgagggtcgtccaagagaaggcgcgaaaaagcatc |
| | | gagttcctggagcagcgatttgagagcagggactcatgcctgagagccctcacgtctcgggggaacgagctgg |
| | | agtccatcgctttccgaagtgaagacattacgggccaacttgggaataatgatgacctcatcttgtcctgctgcctg |
| | | cactactgcaaggacaaggctaaggacttcatgcctgcctccaaggaggagcctatccgattgttgagggaagta |
| | | gtacttttgacggacgaccgcaacctccgggtaaaggcgctgactcgaaatgtcccagtaagggatataccggc |
| | | gttccttacatgggctcaagtagggagtggaagtgagacaccgggaacctcagagagcgccacgccagaaagc |
| | | tacccatacgatgttccagattacgctcatgaatgccgcgtgtgcggcgtgaccgaagtgggcctgagcgcgtat |
| | | gcgaaacatattagcggccagctgcat |
| 12 | ZNF2_HA_ | atgcagatggagctcgaaatcaggccgctgttcctcgtgccggacactaatggttttatagatcacttggcgtcctt |
| | XTEN_PIN_ | ggctagacttctggaaagccgaaagtatatattggtagtgccgttgattgtaattaacgaattggatgggttggcga |
| | 5′ | aaggacaagagactgatcacagagcaggaggctacgcgagggtcgtccaagagaaggcgcgaaaaagcatc |
| | | gagttcctggagcagcgatttgagagcagggactcatgcctgagagccctcacgtctcgggggaacgagctgg |
| | | agtccatcgctttccgaagtgaagacattacgggccaacttgggaataatgatgacctcatcttgtcctgctgcctg |
| | | cactactgcaaggacaaggctaaggacttcatgcctgcctccaaggaggagcctatccgattgttgagggaagta |
| | | gtacttttgacggacgaccgcaacctccgggtaaaggcgctgactcgaaatgtcccagtaagggatataccggc |
| | | gttccttacatgggctcaagtagggagtggaagtgagacaccgggaacctcagagagcgccacgccagaaagc |
| | | tatcgctgctggtggcatggctgcagcctgatttttggcgtggtggatcatctgaaacagcatctgctgaccgatcat |
| | | acccatacgatgttccagattacgct |
| 13 | ZNF1&2HA_ | atgcagatggagctcgaaatcaggccgctgttcctcgtgccggacactaatggttttatagatcacttggcgtcctt |
| | XTEN_ | ggctagacttctggaaagccgaaagtatatattggtagtgccgttgattgtaattaacgaattggatgggttggcga |
| | PIN_5′ | aaggacaagagactgatcacagagcaggaggctacgcgagggtcgtccaagagaaggcgcgaaaaagcatc |
| | | gagttcctggagcagcgatttgagagcagggactcatgcctgagagccctcacgtctcgggggaacgagctgg |
| | | agtccatcgctttccgaagtgaagacattacgggccaacttgggaataatgatgacctcatcttgtcctgctgcctg |
| | | cactactgcaaggacaaggctaaggacttcatgcctgcctccaaggaggagcctatccgattgttgagggaagta |
| | | gtacttttgacggacgaccgcaacctccgggtaaaggcgctgactcgaaatgtcccagtaagggatataccggc |
| | | gttccttacatgggctcaagtagggagtggaagtgagacaccgggaacctcagagagcgccacgccagaaagc |
| | | catgaatgccgcgtgtgcggcgtgaccgaagtgggcctgagcgcgtatgcgaaacatattagcggccagctgc |
| | | atggcagcggaagtgggcatgaatgccgcgtgtgcggcgtgaccgaagtgggcctgagcgcgtatgcgaaac |
| | | atattagcggccagctgcatacccatacgatgttccagattacgct |
| 14 | ZNF1_HA_ | Catgaatgccgcgtgtgcggcgtgaccgaagtgggcctgagcgcgtatgcgaaacatattagcggccagctgc |
| | XTEN_PIN_ | atggcagcggaagtgggcatgaatgccgcgtgtgcggcgtgaccgaagtgggcctgagcgcgtatgcgaaac |
| | 3′ | atattagcggccagctgcattacccatacgatgttccagattacgctatgcagatggagctcgaaatcaggccgct |
| | | gttcctcgtgccggacactaatggttttatagatcacttggcgtccttggtagacttctggaaagccgaaagtatatat |
| | | tggtagtgccgttgattgtaattaacgaattggatgggttggcgaaaggacaagagactgatcacagagcaggag |
| | | gctacgcgagggtcgtccaagagaaggcgcgaaaaagcatcgagttcctggagcagcgatttgagagcaggg |
| | | actcatgcctgagagccctcacgtctcgggggaacgagctggagtccatcgctttccgaagtgaagacattacgg |
| | | gccaacttgggaataatgatgacctcatcttgtcctgctgcctgcactactgcaaggacaaggctaaggacttcatg |
| | | cctgcctccaaggaggagcctatccgattgttgagggaagtagtacttttgacggacgaccgcaacctccgggta |
| | | aaggcgctgactcgaaatgtcccagtaagggatataccggcgttccttacatgggctcaagtagggagtggaagt |
| | | gagacaccgggaacctcagagagcgccacgccagaaagc |
| 15 | ZNF2_HA_ | atgcagatggagctcgaaatcaggccgctgttcctcgtgccggacactaatggttttatagatcacttggcgtcctt |
| | XTEN_PIN_ | ggctagacttctggaaagccgaaagtatatattggtagtgccgttgattgtaattaacgaattggatgggttggcga |
| | 3′ | aaggacaagagactgatcacagagcaggaggctacgcgagggtcgtccaagagaaggcgcgaaaaagcatc |
| | | gagttcctggagcagcgatttgagagcagggactcatgcctgagagccctcacgtctcgggggaacgagctgg |
| | | agtccatcgctttccgaagtgaagacattacgggccaacttgggaataatgatgacctcatcttgtcctgctgcctg |
| | | cactactgcaaggacaaggctaaggacttcatgcctgcctccaaggaggagcctatccgattgttgagggaagta |
| | | gtacttttgacggacgaccgcaacctccgggtaaaggcgctgactcgaaatgtcccagtaagggatataccggc |
| | | gttccttacatgggctcaagtagggagtggaagtgagacaccgggaacctcagagagcgccacgccagaaagc |
| 16 | ZNF1&2HA_ | atgcagatggagctcgaaatcaggccgctgttcctcgtgccggacactaatggttttatagatcacttggcgtcctt |
| | XTEN_P | ggctagacttctggaaagccgaaagtatatattggtagtgccgttgattgtaattaacgaattggatgggttggcga |
| | IN_3′ | aaggacaagagactgatcacagagcaggaggctacgcgagggtcgtccaagagaaggcgcgaaaaagcatc |
| | | gagttcctggagcagcgatttgagagcagggactcatgcctgagagccctcacgtctcgggggaacgagctgg |
| | | agtccatcgctttccgaagtgaagacattacgggccaacttgggaataatgatgacctcatcttgtcctgctgcctg |
| | | cactactgcaaggacaaggctaaggacttcatgcctgcctccaaggaggagcctatccgattgttgagggaagta |
| | | gtacttttgacggacgaccgcaacctccgggtaaaggcgctgactcgaaatgtcccagtaagggatataccggc |
| | | gttccttacatgggctcaagtagggagtggaagtgagacaccgggaacctcagagagcgccacgccagaaagc |
| 17 | ZNF1&2HA_ | catgaatgccgcgtgtgcggcgtgaccgaagtgggcctgagcgcgtatgcgaaacatattagcggccagctgc |
| | 5′ | atggcagcggaagtgggtatcgctgctggtggcatggctgcagcctgatttttggcgtggtggatcatctgaaaca |
| | | gcatctgctgaccgatcatggatccggacctaagaaaaagaggaaggtggcggccgcttacccatacgatgttc |
| | | cagattacgctga |
| 18 | ZNF_1_HA_ | atgcagatggagctcgaaatcaggccgctgttcctcgtgccggacactaatggttttatagatcacttggcgtcctt |
| | PIN_XTEN_ | ggctagacttctggaaagccgaaagtatatattggtagtgccgttgattgtaattaacgaattggatgggttggcga |
| | 5′ | aaggacaagagactgatcacagagcaggaggctacgcgagggtcgtccaagagaaggcgcgaaaaagcatc |
| | | gagttcctggagcagcgatttgagagcagggactcatgcctgagagccctcacgtctcgggggaacgagctgg |
| | | agtccatcgctttccgaagtgaagacattacgggccaacttgggaataatgatgacctcatcttgtcctgctgcctg |
| | | cactactgcaaggacaaggctaaggacttcatgcctgcctccaaggaggagcctatccgattgttgagggaagta |
| | | gtacttttgacggacgaccgcaacctccgggtaaaggcgctgactcgaaatgtcccagtaagggatataccggc |
| | | gttccttacatgggctcaagtagggagtggaagtgagacaccgggaacctcagagagcgccacgccagaaagc |
| | | catgaatgccgcgtgtgcggcgtgaccgaagtgggcctgagcgcgtatgcgaaacatattagcggccagctgc |
| | | at |
| 19 | ZNF_1_HA_ | catgaatgccgcgtgtgcggcgtgaccgaagtgggcctgagcgcgtatgcgaaacatattagcggccagctgc |
| | PIN_XTEN_ | atatgcagatggagctcgaaatcaggccgctgttcctcgtgccggacactaatggttttatagatcacttggcgtcct |
| | 3′ | tggctagacttctggaaagccgaaagtatatattggtagtgccgttgattgtaattaacgaattggatgggttggcga |
| | | aaggacaagagactgatcacagagcaggaggctacgcgagggtcgtccaagagaaggcgcgaaaaagcatc |
| | | gagttcctggagcagcgatttgagagcagggactcatgcctgagagccctcacgtctcgggggaacgagctgg |
| | | agtccatcgctttccgaagtgaagacattacgggccaacttgggaataatgatgacctcatcttgtcctgctgcctg |
| | | cactactgcaaggacaaggctaaggacttcatgcctgcctccaaggaggagcctatccgattgttgagggaagta |
| | | gtacttttgacggacgaccgcaacctccgggtaaaggcgctgactcgaaatgtcccagtaagggatataccggc |
| | | gttccttacatgggctcaagtagggagtggaagtgagacaccgggaacctcagagagcgccacgccagaaagc |
| 20 | ZNF_2_HA_ | atgcagatggagctcgaaatcaggccgctgttcctcgtgccggacactaatggttttatagatcacttggcgtcctt |
| | PIN_XTEN_ | ggctagacttctggaaagccgaaagtatatattggtagtgccgttgattgtaattaacgaattggatgggttggcga |
| | 5′ | aaggacaagagactgatcacagagcaggaggctacgcgagggtcgtccaagagaaggcgcgaaaaagcatc |
| | | gagttcctggagcagcgatttgagagcagggactcatgcctgagagccctcacgtctcgggggaacgagctgg |
| | | agtccatcgctttccgaagtgaagacattacgggccaacttgggaataatgatgacctcatcttgtcctgctgcctg |
| | | cactactgcaaggacaaggctaaggacttcatgcctgcctccaaggaggagcctatccgattgttgagggaagta |
| | | gtacttttgacggacgaccgcaacctccgggtaaaggcgctgactcgaaatgtcccagtaagggatataccggc |
| | | gttccttacatgggctcaagtagggagtggaagtgagacaccgggaacctcagagagcgccacgccagaaagc |
| | | tatcgctgctggtggcatggctgcagcctgatttttggcgtggtggatcatctgaaacagcatctgctgaccgatcat |
| | | ggatccggacctaagaaaaagaggaaggtggcggccgcttacccatacgatgttccagattacgct |
| 21 | ZNF_2_HA_ | tatcgctgctggtggcatggctgcagcctgatttttggcgtggtggatcatctgaaacagcatctgctgaccgatcat |
| | PIN_XTEN_ | ggatccggacctaagaaaaagaggaaggtggcggccgcttacccatacgatgttccagattacgctgaatgcag |
| | 3′ | atggagctcgaaatcaggccgctgttcctcgtgccggacactaatggttttatagatcacttggcgtccttggctag |
| | | acttctggaaagccgaaagtatatattggtagtgccgttgattgtaattaacgaattggatgggttggcgaaaggac |
| | | aagagactgatcacagagcaggaggctacgcgagggtcgtccaagagaaggcgcgaaaaagcatcgagttcc |
| | | tggagcagcgatttgagagcagggactcatgcctgagagccctcacgtctcgggggaacgagctggagtccat |
| | | cgctttccgaagtgaagacattacgggccaacttgggaataatgatgacctcatcttgtcctgctgcctgcactact |
| | | gcaaggacaaggctaaggacttcatgcctgcctccaaggaggagcctatccgattgttgagggaagtagtactttt |
| | | gacggacgaccgcaacctccgggtaaaggcgctgactcgaaatgtcccagtaagggatataccggcgttcctta |
| | | catgggctcaagtagggagtggaagtgagacaccgggaacctcagagagcgccacgccagaaagc |
| 22 | ZNF1&2_HA_ | atgcagatggagctcgaaatcaggccgctgttcctcgtgccggacactaatggttttatagatcacttggcgtcctt |
| | PIN_XTEN_ | ggctagacttctggaaagccgaaagtatatattggtagtgccgttgattgtaattaacgaattggatgggttggcga |
| | 5′ | aaggacaagagactgatcacagagcaggaggctacgcgagggtcgtccaagagaaggcgcgaaaaagcatc |
| | | gagttcctggagcagcgatttgagagcagggactcatgcctgagagccctcacgtctcgggggaacgagctgg |
| | | agtccatcgctttccgaagtgaagacattacgggccaacttgggaataatgatgacctcatcttgtcctgctgcctg |
| | | cactactgcaaggacaaggctaaggacttcatgcctgcctccaaggaggagcctatccgattgttgagggaagta |
| | | gtacttttgacggacgaccgcaacctccgggtaaaggcgctgactcgaaatgtcccagtaagggatataccggc |
| | | gttccttacatgggctcaagtagggagtggaagtgagacaccgggaacctcagagagcgccacgccagaaagc |
| | | catgaatgccgcgtgtgcggcgtgaccgaagtgggcctgagcgcgtatgcgaaacatattAgcggccagctgc |
| | | atggcagcggaagtgggtatcgctgctggtggcatggctgcagcctgatttttggcgtggtggatcatctgaaaca |
| | | gcatctgctgaccgatcatggatccggacctaagaaaaagaggaaggtggcggccgcttacccatacgatgttc |
| | | cagattacgctga |
| 23 | ZNF1&2_HA_ | catgaatgccgcgtgtgcggcgtgaccgaagtgggcctgagcgcgtatgcgaaacatattagcggccagctgc |
| | PIN_XTEN_ | atggcagcggaagtgggtatcgctgctggtggcatggctgcagcctgatttttggcgtggtggatcatctgaaaca |
| | 3′ | gcatctgctgaccgatcatggatccggacctaagaaaaagaggaaggtggcggccgcttacccatacgatgttc |
| | | cagattacgctgaatgcagatggagctcgaaatcaggccgctgttcctcgtgccggacactaatggttttatagatc |
| | | acttggcgtccttggctagacttctggaaagccgaaagtatatattggtagtgccgttgattgtaattaacgaattgga |
| | | tgggttggcgaaaggacaagagactgatcacagagcaggaggctacgcgagggtcgtccaagagaaggcgc |
| | | gaaaaagcatcgagttcctggagcagcgatttgagagcagggactcatgcctgagagccctcacgtctcggggg |
| | | aacgagctggagtccatcgctttccgaagtgaagacattacgggccaacttgggaataatgatgacctcatcttgtc |
| | | ctgctgcctgcactactgcaaggacaaggctaaggacttcatgcctgcctccaaggaggagcctatccgattgttg |
| | | agggaagtagtacttttgacggacgaccgcaacctccgggtaaaggcgctgactcgaaatgtcccagtaaggga |
| | | tataccggcgttccttacatgggctcaagtagggagtggaagtgagacaccgggaacctcagagagcgccacg |
| | | ccagaaagc |
| 24 | ZNF1_gene | gacattgattattgactagttattaatagtaatcaattacggggtcattagttcatagcccatatatggagttccgcgtta |
| | delivery | cataacttacggtaaatggcccgcctggctgaccgcccaacgacccccgcccattgacgtcaataatgacgtatg |
| | vehicle | ttcccatagtaacgccaatagggactttccattgacgtcaatgggtggagtatttacggtaaactgcccacttggca |
| | insert | gtacatcaagtgtatcatatgccaagtacgccccctattgacgtcaatgacggtaaatggcccgcctggcattatgc |
| | | ccagtacatgaccttatgggactttcctacttggcagtacatctacgtattagtcatcgctattaccatggtgatgcgg |
| | | ttttggcagtacatcaatgggcgtggatagcggtttgactcacggggatttccaagtctccaccccattgacgtcaat |
| | | gggagtttgttttggcaccaaaatcaacgggactttccaaaatgtcgtaacaactccgccccattgacgcaaatgg |
| | | gcggtaggcgtgtacggtgggaggtctatataagcagagctgaccggttctagagcgctgccaccatgagcccc |
| | | aagaagaagagaaaggtggaggccagcatgcagatggagctcgaaatcaggccgctgttcctcgtgccggac |
| | | actaatggttttatagatcacttggcgtccttggctagacttctggaaagccgaaagtatatattggtagtgccgttga |
| | | ttgtaattaacgaattggatgggttggcgaaaggacaagagactgatcacagagcaggaggctacgcgagggtc |
| | | gtccaagagaaggcgcgaaaaagcatcgagttcctggagcagcgatttgagagcagggactcatgcctgagag |
| | | ccctcacgtctcgggggaacgagctggagtccatcgctttccgaagtgaagacattacgggccaacttgggaata |
| | | atgatgacctcatcttgtcctgctgcctgcactactgcaaggacaaggctaaggacttcatgcctgcctccaagga |
| | | ggagcctatccgattgttgagggaagtagtacttttgacggacgaccgcaacctccgggtaaaggcgctgactcg |
| | | aaatgtcccagtaagggatataccggcgttccttacatgggctcaagtagggagtggaagtgagacaccgggaa |
| | | cctcagagagcgccacgccagaaagccatgaatgccgcgtgtgcggcgtgaccgaagtgggcctgagcgcgt |
| | | atgcgaaacatattagcggccagctgcatggacctaagaaaaagaggaaggtggcggccgcttacccatacgat |
| | | gttccagattacgctgacgtggaattctaacaattgttgttgttaacttgtttattgcagcttataatggttacaaataaa |
| | | gcaatagcatcacaaatttcacaaataaagcatttttttcactgcattctagttgtggtttgtccaaactcatcaatgtat |
| | | cttaagatatctagatctcgaggtaaccacgtgcggaccgagcggccgc |
| 25 | ZNF2_gene | gacattgattattgactagttattaatagtaatcaattacggggtcattagttcatagcccatatatggagttccgcgtta |
| | delivery | cataacttacggtaaatggcccgcctggctgaccgcccaacgacccccgcccattgacgtcaataatgacgtatg |
| | vehicle | ttcccatagtaacgccaatagggactttccattgacgtcaatgggtggagtatttacggtaaactgcccacttggca |
| | insert | gtacatcaagtgtatcatatgccaagtacgccccctattgacgtcaatgacggtaaatggcccgcctggcattatgc |
| | | ccagtacatgaccttatgggactttcctacttggcagtacatctacgtattagtcatcgctattaccatggtgatgcgg |
| | | ttttggcagtacatcaatgggcgtggatagcggtttgactcacggggatttccaagtctccaccccattgacgtcaat |
| | | gggagtttgttttggcaccaaaatcaacgggactttccaaaatgtcgtaacaactccgccccattgacgcaaatgg |
| | | gcggtaggcgtgtacggtgggaggtctatataagcagagctgaccggttctagagcgctgccaccatgagcccc |
| | | aagaagaagagaaaggtggaggccagcatgcagatggagctcgaaatcaggccgctgttcctcgtgccggac |
| | | actaatggttttatagatcacttggcgtccttggctagacttctggaaagccgaaagtatatattggtagtgccgttga |
| | | ttgtaattaacgaattggatgggttggcgaaaggacaagagactgatcacagagcaggaggctacgcgagggtc |
| | | gtccaagagaaggcgcgaaaaagcatcgagttcctggagcagcgatttgagagcagggactcatgcctgagag |
| | | ccctcacgtctcgggggaacgagctggagtccatcgctttccgaagtgaagacattacgggccaacttgggaata |
| | | atgatgacctcatcttgtcctgctgcctgcactactgcaaggacaaggctaaggacttcatgcctgcctccaagga |
| | | ggagcctatccgattgttgagggaagtagtacttttgacggacgaccgcaacctccgggtaaaggcgctgactcg |
| | | aaatgtcccagtaagggatataccggcgttccttacatgggctcaagtagggagtggaagtgagacaccgggaa |
| | | cctcagagagcgccacgccagaaagctatcgctgctggtggcatggctgcagcctgatttttggcgtggtggatc |
| | | atctgaaacagcatctgctgaccgatcatggatccggacctaagaaaaagaggaaggtggcggccgcttaccca |
| | | tacgatgttccagattacgctga |
| 26 | ZNF1&2_gene | gacattgattattgactagttattaatagtaatcaattacggggtcattagttcatagcccatatatggagttccgcgtta |
| | delivery | cataacttacggtaaatggcccgcctggctgaccgcccaacgacccccgcccattgacgtcaataatgacgtatg |
| | vehicle | ttcccatagtaacgccaatagggactttccattgacgtcaatgggtggagtatttacggtaaactgcccacttggca |
| | insert | gtacatcaagtgtatcatatgccaagtacgccccctattgacgtcaatgacggtaaatggcccgcctggcattatgc |
| | | ccagtacatgaccttatgggactttcctacttggcagtacatctacgtattagtcatcgctattaccatggtgatgcgg |
| | | ttttggcagtacatcaatgggcgtggatagcggtttgactcacggggatttccaagtctccaccccattgacgtcaat |
| | | gggagtttgttttggcaccaaaatcaacgggactttccaaaatgtcgtaacaactccgccccattgacgcaaatgg |
| | | gcggtaggcgtgtacggtgggaggtctatataagcagagctgaccggttctagagcgctgccaccatgagcccc |
| | | aagaagaagagaaaggtggaggccagcatgcagatggagctcgaaatcaggccgctgttcctcgtgccggac |
| | | actaatggttttatagatcacttggcgtccttggctagacttctggaaagccgaaagtatatattggtagtgccgttga |
| | | ttgtaattaacgaattggatgggttggcgaaaggacaagagactgatcacagagcaggaggctacgcgagggtc |
| | | gtccaagagaaggcgcgaaaaagcatcgagttcctggagcagcgatttgagagcagggactcatgcctgagag |
| | | ccctcacgtctcgggggaacgagctggagtccatcgctttccgaagtgaagacattacgggccaacttgggaata |
| | | atgatgacctcatcttgtcctgctgcctgcactactgcaaggacaaggctaaggacttcatgcctgcctccaagga |
| | | ggagcctatccgattgttgagggaagtagtacttttgacggacgaccgcaacctccgggtaaaggcgctgactcg |
| | | aaatgtcccagtaagggatataccggcgttccttacatgggctcaagtagggagtggaagtgagacaccgggaa |
| | | cctcagagagcgccacgccagaaagccatgaatgccgcgtgtgcggcgtgaccgaagtgggcctgagcgcgt |
| | | atgcgaaacatattAgcggccagctgcatggcagcggaagtgggtatcgctgctggtggcatggctgcagcct |
| | | gatttttggcgtggtggatcatctgaaacagcatctgctgaccgatcatggatccggacctaagaaaaagaggaa |
| | | ggtggcggccgcttacccatacgatgttccagattacgctgacgtggaattctaacaattgttgttgttaacttgtttat |
| | | tgcagcttataatggttacaaataaagcaatagcatcacaaatttcacaaataaagcatttttttcactgcattctagttg |
| | | tggtttgtccaaactcatcaatgtatctta |
| 27 | ZNF1_AAV | cctgcaggcagctgcgcgctcgctcgctcactgaggccgcccgggcgtcgggcgacctttggtcgcccggcct |
| | Insert | cagtgagcgagcgagcgcgcagagagggagtgggcggccgcggccgcacgcgtgacattgattattgactag |
| | | ttattaatagtaatcaattacggggtcattagttcatagcccatatatggagttccgcgttacataacttacggtaaatg |
| | | gcccgcctggctgaccgcccaacgacccccgcccattgacgtcaataatgacgtatgttcccatagtaacgccaa |
| | | tagggactttccattgacgtcaatgggtggagtatttacggtaaactgcccacttggcagtacatcaagtgtatcata |
| | | tgccaagtacgccccctattgacgtcaatgacggtaaatggcccgcctggcattatgcccagtacatgaccttatg |
| | | ggactttcctacttggcagtacatctacgtattagtcatcgctattaccatggtgatgcggttttggcagtacatcaatg |
| | | ggcgtggatagcggtttgactcacggggatttccaagtctccaccccattgacgtcaatgggagtttgttttggcac |
| | | caaaatcaacgggactttccaaaatgtcgtaacaactccgccccattgacgcaaatgggcggtaggcgtgtacgg |
| | | tgggaggtctatataagcagagctgaccggttctagagcgctgccaccatgagccccaagaagaagagaaagg |
| | | tggaggccagcatgcagatggagctcgaaatcaggccgctgttcctcgtgccggacactaatggttttatagatca |
| | | cttggcgtccttggctagacttctggaaagccgaaagtatatattggtagtgccgttgattgtaattaacgaattggat |
| | | gggttggcgaaaggacaagagactgatcacagagcaggaggctacgcgagggtcgtccaagagaaggcgcg |
| | | aaaaagcatcgagttcctggagcagcgatttgagagcagggactcatgcctgagagccctcacgtctcggggga |
| | | acgagctggagtccatcgctttccgaagtgaagacattacgggccaacttgggaataatgatgacctcatcttgtcc |
| | | tgctgcctgcactactgcaaggacaaggctaaggacttcatgcctgcctccaaggaggagcctatccgattgttga |
| | | gggaagtagtacttttgacggacgaccgcaacctccgggtaaaggcgctgactcgaaatgtcccagtaagggat |
| | | ataccggcgttccttacatgggctcaagtagggagtggaagtgagacaccgggaacctcagagagcgccacgc |
| | | cagaaagccatgaatgccgcgtgtgcggcgtgaccgaagtgggcctgagcgcgtatgcgaaacatattAgcg |
| | | gccagctgcatggacctaagaaaaagaggaaggtggcggccgcttacccatacgatgttccagattacgctgac |
| | | gtggaattctaacaattgttgttgttaacttgtttattgcagcttataatggttacaaataaagcaatagcatcacaaatt |
| | | tcacaaataaagcatttttttcactgcattctagttgtggtttgtccaaactcatcaatgtatcttaagatatctagatct |
| | | cgaggtaaccacgtgcggaccgagcggccgcaggaacccctagtgatggagttggccactccctctctgcgcgct |
| | | cgctcgctcactgaggccgggcgaccaaaggtcgcccgacgcccgggctttgcccgggcggcctcagtgagc |
| | | gagcgagcgcgcagctgcctgcagg |
| 28 | ZNF3_AAV | cctgcaggcagctgcgcgctcgctcgctcactgaggccgcccgggcgtcgggcgacctttggtcgcccggcct |
| | Insert: | cagtgagcgagcgagcgcgcagagagggagtgggcggccgcggccgcacgcgtgacattgattattgactag |
| | | ttattaatagtaatcaattacggggtcattagttcatagcccatatatggagttccgcgttacataacttacggtaaatg |
| | | gcccgcctggctgaccgcccaacgacccccgcccattgacgtcaataatgacgtatgttcccatagtaacgccaa |
| | | tagggactttccattgacgtcaatgggtggagtatttacggtaaactgcccacttggcagtacatcaagtgtatcata |
| | | tgccaagtacgccccctattgacgtcaatgacggtaaatggcccgcctggcattatgcccagtacatgaccttatg |
| | | ggactttcctacttggcagtacatctacgtattagtcatcgctattaccatggtgatgcggttttggcagtacatcaatg |
| | | ggcgtggatagcggtttgactcacggggatttccaagtctccaccccattgacgtcaatgggagtttgttttggcac |
| | | caaaatcaacgggactttccaaaatgtcgtaacaactccgccccattgacgcaaatgggcggtaggcgtgtacgg |
| | | tgggaggtctatataagcagagctgaccggttctagagcgctgccaccatgagccccaagaagaagagaaagg |
| | | tggaggccagcatgcagatggagctcgaaatcaggccgctgttcctcgtgccggacactaatggttttatagatca |
| | | cttggcgtccttggctagacttctggaaagccgaaagtatatattggtagtgccgttgattgtaattaacgaattggat |
| | | gggttggcgaaaggacaagagactgatcacagagcaggaggctacgcgagggtcgtccaagagaaggcgcg |
| | | aaaaagcatcgagttcctggagcagcgatttgagagcagggactcatgcctgagagccctcacgtctcggggga |
| | | acgagctggagtccatcgctttccgaagtgaagacattacgggccaacttgggaataatgatgacctcatcttgtcc |
| | | tgctgcctgcactactgcaaggacaaggctaaggacttcatgcctgcctccaaggaggagcctatccgattgttga |
| | | gggaagtagtacttttgacggacgaccgcaacctccgggtaaaggcgctgactcgaaatgtcccagtaagggat |
| | | ataccggcgttccttacatgggctcaagtagggagtggaagtgagacaccgggaacctcagagagcgccacgc |
| | | cagaaagctatcgctgctggtggcatggctgcagcctgatttttggcgtggtggatcatctgaaacagcatctgctg |
| | | accgatcatggatccggacctaagaaaaagaggaaggtggcggccgcttacccatacgatgttccagattacgct |
| | | gacgtggaattctaacaattgttgttgttaacttgtttattgcagcttataatggttacaaataaagcaatagcatcacaa |
| | | atttcacaaataaagcatttttttcactgcattctagttgtggtttgtccaaactcatcaatgtatcttaagatatctaga |
| | | tctcgaggtaaccacgtgcggaccgagcggccgcaggaacccctagtgatggagttggccactccctctctgcgc |
| | | gctcgctcgctcactgaggccgggcgaccaaaggtcgcccgacgcccgggctttgcccgggcggcctcagtg |
| | | agcgagcgagcgcgcagctgcctgcagg |
| 29 | ZNF1&2_ | cctgcaggcagctgcgcgctcgctcgctcactgaggccgcccgggcgtcgggcgacctttggtcgcccggcct |
| | AAV Insert: | cagtgagcgagcgagcgcgcagagagggagtgggcggccgcggccgcacgcgtgacattgattattgactag |
| | | ttattaatagtaatcaattacggggtcattagttcatagcccatatatggagttccgcgttacataacttacggtaaatg |
| | | gcccgcctggctgaccgcccaacgacccccgcccattgacgtcaataatgacgtatgttcccatagtaacgccaa |
| | | tagggactttccattgacgtcaatgggtggagtatttacggtaaactgcccacttggcagtacatcaagtgtatcata |
| | | tgccaagtacgccccctattgacgtcaatgacggtaaatggcccgcctggcattatgcccagtacatgaccttatg |
| | | ggactttcctacttggcagtacatctacgtattagtcatcgctattaccatggtgatgcggttttggcagtacatcaatg |
| | | ggcgtggatagcggtttgactcacggggatttccaagtctccaccccattgacgtcaatgggagtttgttttggcac |
| | | caaaatcaacgggactttccaaaatgtcgtaacaactccgccccattgacgcaaatgggcggtaggcgtgtacgg |
| | | tgggaggtctatataagcagagctgaccggttctagagcgctgccaccatgagccccaagaagaagagaaagg |
| | | tggaggccagcatgcagatggagctcgaaatcaggccgctgttcctcgtgccggacactaatggttttatagatca |
| | | cttggcgtccttggctagacttctggaaagccgaaagtatatattggtagtgccgttgattgtaattaacgaattggat |
| | | gggttggcgaaaggacaagagactgatcacagagcaggaggctacgcgagggtcgtccaagagaaggcgcg |
| | | aaaaagcatcgagttcctggagcagcgatttgagagcagggactcatgcctgagagccctcacgtctcggggga |
| | | acgagctggagtccatcgctttccgaagtgaagacattacgggccaacttgggaataatgatgacctcatcttgtcc |
| | | tgctgcctgcactactgcaaggacaaggctaaggacttcatgcctgcctccaaggaggagcctatccgattgttga |
| | | gggaagtagtacttttgacggacgaccgcaacctccgggtaaaggcgctgactcgaaatgtcccagtaagggat |
| | | ataccggcgttccttacatgggctcaagtagggagtggaagtgagacaccgggaacctcagagagcgccacgc |
| | | cagaaagccatgaatgccgcgtgtgcggcgtgaccgaagtgggcctgagcgcgtatgcgaaacatattAgcg |
| | | gccagctgcatggcagcggaagtgggtatcgctgctggtggcatggctgcagcctgatttttggcgtggtggatc |
| | | atctgaaacagcatctgctgaccgatcatggatccggacctaagaaaaagaggaaggtggcggccgcttaccca |
| | | tacgatgttccagattacgctgacgtggaattctaacaattgttgttgttaacttgtttattgcagcttataatggttaca |
| | | aataaagcaatagcatcacaaatttcacaaataaagcatttttttcactgcattctagttgtggtttgtccaaactcatca |
| | | atgtatcttaagatatctagatctcgaggtaaccacgtgcggaccgagcggccgcaggaacccctagtgatggagt |
| | | tggccactccctctctgcgcgctcgctcgctcactgaggccgggcgaccaaaggtcgcccgacgcccgggcttt |
| | | gcccgggcggcctcagtgagcgagcgagcgcgcagctgcctgcagg |
| 30 | XTEN | SGSETPGTSESATPES |
| | Linker_1 | |
| 31 | XTEN | SGSETPGTSESA |
| | Linker_2 | |
| 32 | XTEN | SGSETPGTSESATPEGGSGGS |
| | Linker_3 | |
| 33 | Linker | (G₄S)n |

1. An isolated nucleic acid encoding a fusion protein, wherein the isolated nucleic acid comprises:
(i) a first sequence encoding a RNA-binding zinc finger domain or a fragment thereof comprising:
an amino acid sequence that is at least 90% identical to the sequence of
(SEQ ID NO: 1)
HECRVCGVTEVGLSAYAKHISGQLH,
or
an amino acid sequence that is at least 90% identical to the sequence of
(SEQ ID NO: 2)
YRCWWHGCSLIFGVVDHLKQHLLTDH;
and
(ii) a second sequence encoding a fusion partner.

2. The isolated nucleic acid of claim 1, wherein the first sequence further comprises an amino acid sequence encoding a second RNA-binding zinc finger domain.

3. The isolated nucleic acid of claim 2, wherein:
(i) a first RNA-binding zinc finger domain comprises SEQ ID NO: 1 and the second RNA-binding zinc finger domain comprises SEQ ID NO: 2;
(ii) the first RNA-binding zinc finger domain comprises SEQ ID NO: 2 and the second RNA-binding zinc finger domain comprises SEQ ID NO: 1;
(iii) the first RNA-binding zinc finger domain comprises SEQ ID NO: 1 and the second RNA-binding zinc finger domain comprises SEQ ID NO: 1; or
(iv) the first RNA-binding zinc finger domain comprises SEQ ID NO: 2 and the second RNA-binding zinc finger domain comprises SEQ ID NO: 2.

4. The isolated nucleic acid of claim 3, wherein the first RNA-binding zinc finger domain is directly adjacent to the second RNA-binding zinc finger domain.

5. The isolated nucleic acid of claim 3, wherein the isolated nucleic acid further comprises a sequence encoding a linker positioned between the first RNA-binding zinc finger domain and the second RNA-binding zinc finger domain.

6. The isolated nucleic acid of claim 1, wherein the first sequence encoding the RNA-binding zinc finger domain comprises three or more RNA-binding zinc finger domains.

7. The isolated nucleic acid of claim 1, wherein the first sequence is directly adjacent to the second sequence.

8. The isolated nucleic acid of claim 1, wherein the isolated nucleic acid further comprises a sequence encoding a linker positioned between the first sequence and the second sequence.

9. The isolated nucleic acid of claim 1, wherein the fusion partner comprises a RNA degrading enzyme, wherein the RNA degrading enzyme comprises an endonuclease, a 5′ exonuclease, or a 3′ exonuclease.

10. (canceled)

11. The isolated nucleic acid of claim 9, wherein the endonuclease comprises a human endonuclease, wherein the human endonuclease cleaves single stranded RNA.

12. The isolated nucleic acid of claim 11, wherein the endonuclease comprises a PIN (PilT N-terminal domain) RNA endonuclease domain or active fragment thereof.

13. A gene delivery vector comprising the isolated nucleic acid of claim 1.

14. The gene delivery vector of claim 13, wherein the gene delivery vector is selected from the group consisting of an adenoviral vector, an adeno associated viral (AAV) vector, a lentiviral vector, and a retroviral vector, wherein the gene delivery vector is an AAV9 vector.

15. (canceled)

16. A pharmaceutical composition comprising the isolated nucleic acid of claim 1.

17. A method of decreasing a level of RNA having a G₄C₂ hexanucleotide repeat in the central nervous system (CNS) of a subject in need thereof, comprising administering to the subject an effective amount of the isolated nucleic acid of claim 1.

18. A method of treating a subject having a G₄C₂ or C₄G₂ hexanucleotide repeat-associated disease or disorder comprising administering to the subject a therapeutically effective amount of the isolated nucleic acid of claim 1.

19. The method of claim 17, wherein the subject is previously diagnosed or identified as having a G₄C₂ or a C₄G₂ hexanucleotide repeat-associated disease or disorder, wherein the G₄C₂ hexanucleotide repeat-associated disease or disorder is frontotemporal dementia (FTD) or amyotrophic lateral sclerosis (ALS).

20. (canceled)

21. A pharmaceutical composition comprising the gene delivery vector of claim 13.

22. A method of decreasing a level of RNA having a G₄C₂ hexanucleotide repeat in the central nervous system (CNS) of a subject in need thereof, comprising administering to the subject an effective amount of the gene delivery vector of claim 13.

23. A method of treating a subject having a G₄C₂ or C₄G₂ hexanucleotide repeat-associated disease or disorder comprising administering to the subject a therapeutically effective amount of the gene delivery vector of claim 13.


SEQUENCE LISTING (33 sequences)

SEQ ID NO: 1 - protein, 25 aa, Homo sapiens
HECRVCGVTEVGLSAYAKHISGQLH

SEQ ID NO: 2 - protein, 26 aa, Homo sapiens
YRCWWHGCSLIFGVVDHLKQHLLTDH

SEQ ID NO: 3 - DNA, 75 bp, Homo sapiens
catgaatgccgcgtgtgcggcgtgaccgaagtgggcctgagcgcgtatgcgaaacatattagcggccagctgcat

SEQ ID NO: 4 - DNA, 78 bp, Homo sapiens
tatcgctgctggtggcatggctgcagcctgatttttggcgtggtggatcatctgaaacagcatctgctgaccgatcat

SEQ ID NO: 5 - DNA, 102 bp, Artificial Sequence
  ZNF with Tag
catgaatgccgcgtgtgcggcgtgaccgaagtgggcctgagcgcgtatgcgaaacatattagcggccagctgcattacccatacgatgttccagattacgct

SEQ ID NO: 6 - DNA, 105 bp, Artificial Sequence
  ZNF with Tag
tatcgctgctggtggcatggctgcagcctgatttttggcgtggtggatcatctgaaacagcatctgctgaccgatcattacccatacgatgttccagattacgct

SEQ ID NO: 7 - DNA, 102 bp, Artificial Sequence
  ZNF with Tag
tacccatacgatgttccagattacgctcatgaatgccgcgtgtgcggcgtgaccgaagtgggcctgagcgcgtatgcgaaacatattagcggccagctgcat

SEQ ID NO: 8 - DNA, 104 bp, Artificial Sequence
  ZNF with Tag
tatcgctgctggtggcatggctgcagcctgatttttggcgtggtggatcatctgaaacagcatctgctgaccgatcatacccatacgatgttccagattacgct

SEQ ID NO: 9 - DNA, 192 bp, Artificial Sequence
  ZNF with Tag
catgaatgccgcgtgtgcggcgtgaccgaagtgggcctgagcgcgtatgcgaaacatattagcggccagctgcatggcagcggaagtgggcatgaatgccgcgtgtgcggcgtgaccgaagtgggcctgagcgcgtatgcgaaacatattagcggccagctgcattacccatacgatgttccagattacgct

SEQ ID NO: 10 - DNA, 191 bp, Artificial Sequence
  ZNF with Tag
catgaatgccgcgtgtgcggcgtgaccgaagtgggcctgagcgcgtatgcgaaacatattagcggccagctgcatggcagcggaagtgggcatgaatgccgcgtgtgcggcgtgaccgaagtgggcctgagcgcgtatgcgaaacatattagcggccagctgcatacccatacgatgttccagattacgct

SEQ ID NO: 11 - DNA, 699 bp, Artificial Sequence
  ZNF fusion protein
atgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagctacccatacgatgttccagattacgctcatgaatgccgcgtgtgcggcgtgaccgaagtgggcctgagcgcgtatgcgaaacatattagcggccagctgcat

SEQ ID NO: 12 - DNA, 701 bp, Artificial Sequence
  ZNF fusion protein
atgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagctatcgctgctggtggcatggctgcagcctgatttttggcgtggtggatcatctgaaacagcatctgctgaccgatcatacccatacgatgttccagattacgct

SEQ ID NO: 13 - DNA, 788 bp, Artificial Sequence
  ZNF fusion protein
atgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagccatgaatgccgcgtgtgcggcgtgaccgaagtgggcctgagcgcgtatgcgaaacatattagcggccagctgcatggcagcggaagtgggcatgaatgccgcgtgtgcggcgtgaccgaagtgggcctgagcgcgtatgcgaaacatattagcggccagctgcatacccatacgatgttccagattacgct

SEQ ID NO: 14 - DNA, 788 bp, Artificial Sequence
  ZNF fusion protein
catgaatgccgcgtgtgcggcgtgaccgaagtgggcctgagcgcgtatgcgaaacatattagcggccagctgcatggcagcggaagtgggcatgaatgccgcgtgtgcggcgtgaccgaagtgggcctgagcgcgtatgcgaaacatattagcggccagctgcattacccatacgatgttccagattacgctatgcagatggagctcgaaatcaggccgctgttcctcgtgccggacactaatggttttatagatcacttggcgtccttggtagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagc

SEQ ID NO: 15 - DNA, 597 bp, Artificial Sequence
  ZNF fusion protein
atgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagc

SEQ ID NO: 16 - DNA, 597 bp, Artificial Sequence; identical to SEQ ID NO: 15
  ZNF fusion protein

SEQ ID NO: 17 - DNA, 236 bp, Artificial Sequence
  ZNF fusion protein
catgaatgccgcgtgtgcggcgtgaccgaagtgggcctgagcgcgtatgcgaaacatattagcggccagctgcatggcagcggaagtgggtatcgctgctggtggcatggctgcagcctgatttttggcgtggtggatcatctgaaacagcatctgctgaccgatcatggatccggacctaagaaaaagaggaaggtggcggccgcttacccatacgatgttccagattacgctga

SEQ ID NO: 18 - DNA, 672 bp, Artificial Sequence
  ZNF fusion protein
atgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagccatgaatgccgcgtgtgcggcgtgaccgaagtgggcctgagcgcgtatgcgaaacatattagcggccagctgcat

SEQ ID NO: 19 - DNA, 672 bp, Artificial Sequence
  ZNF fusion protein
catgaatgccgcgtgtgcggcgtgaccgaagtgggcctgagcgcgtatgcgaaacatattagcggccagctgcatatgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagc

SEQ ID NO: 20 - DNA, 741 bp, Artificial Sequence
  ZNF fusion protein
atgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagctatcgctgctggtggcatggctgcagcctgatttttggcgtggtggatcatctgaaacagcatctgctgaccgatcatggatccggacctaagaaaaagaggaaggtggcggccgcttacccatacgatgttccagattacgct

SEQ ID NO: 21 - DNA, 743 bp, Artificial Sequence
  ZNF fusion protein
tatcgctgctggtggcatggctgcagcctgatttttggcgtggtggatcatctgaaacagcatctgctgaccgatcatggatccggacctaagaaaaagaggaaggtggcggccgcttacccatacgatgttccagattacgctgaatgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagc

SEQ ID NO: 22 - DNA, 833 bp, Artificial Sequence
  ZNF fusion protein
atgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagccatgaatgccgcgtgtgcggcgtgaccgaagtgggcctgagcgcgtatgcgaaacatattagcggccagctgcatggcagcggaagtgggtatcgctgctggtggcatggctgcagcctgatttttggcgtggtggatcatctgaaacagcatctgctgaccgatcatggatccggacctaagaaaaagaggaaggtggcggccgcttacccatacgatgttccagattacgctga

SEQ ID NO: 23 - DNA, 833 bp, Artificial Sequence
  ZNF fusion protein
catgaatgccgcgtgtgcggcgtgaccgaagtgggcctgagcgcgtatgcgaaacatattagcggccagctgcatggcagcggaagtgggtatcgctgctggtggcatggctgcagcctgatttttggcgtggtggatcatctgaaacagcatctgctgaccgatcatggatccggacctaagaaaaagaggaaggtggcggccgcttacccatacgatgttccagattacgctgaatgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagc

SEQ ID NO: 24 - DNA, 1572 bp, Artificial Sequence
  ZNF fusion protein
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgaccggttctagagcgctgccaccatgagccccaagaagaagagaaaggtggaggccagcatgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagccatgaatgccgcgtgtgcggcgtgaccgaagtgggcctgagcgcgtatgcgaaacatattagcggccagctgcatggacctaagaaaaagaggaaggtggcggccgcttacccatacgatgttccagattacgctgacgtggaattctaacaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgc

SEQ ID NO: 25 - DNA, 1387 bp, Artificial Sequence
  ZNF fusion protein
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgaccggttctagagcgctgccaccatgagccccaagaagaagagaaaggtggaggccagcatgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagctatcgctgctggtggcatggctgcagcctgatttttggcgtggtggatcatctgaaacagcatctgctgaccgatcatggatccggacctaagaaaaagaggaaggtggcggccgcttacccatacgatgttccagattacgctga

SEQ ID NO: 26 - DNA, 1626 bp, Artificial Sequence
  ZNF fusion protein
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgaccggttctagagcgctgccaccatgagccccaagaagaagagaaaggtggaggccagcatgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagccatgaatgccgcgtgtgcggcgtgaccgaagtgggcctgagcgcgtatgcgaaacatattagcggccagctgcatggcagcggaagtgggtatcgctgctggtggcatggctgcagcctgatttttggcgtggtggatcatctgaaacagcatctgctgaccgatcatggatccggacctaagaaaaagaggaaggtggcggccgcttacccatacgatgttccagattacgctgacgtggaattctaacaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 27 - DNA, 1839 bp, Artificial Sequence
  ZNF fusion protein
cctgcaggcagctgcgcgctcgctcgctcactgaggccgcccgggcgtcgggcgacctttggtcgcccggcctcagtgagcgagcgagcgcgcagagagggagtgggcggccgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgaccggttctagagcgctgccaccatgagccccaagaagaagagaaaggtggaggccagcatgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagccatgaatgccgcgtgtgcggcgtgaccgaagtgggcctgagcgcgtatgcgaaacatattagcggccagctgcatggacctaagaaaaagaggaaggtggcggccgcttacccatacgatgttccagattacgctgacgtggaattctaacaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 28 - DNA, 1848 bp, Artificial Sequence
  ZNF fusion protein
cctgcaggcagctgcgcgctcgctcgctcactgaggccgcccgggcgtcgggcgacctttggtcgcccggcctcagtgagcgagcgagcgcgcagagagggagtgggcggccgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgaccggttctagagcgctgccaccatgagccccaagaagaagagaaaggtggaggccagcatgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagctatcgctgctggtggcatggctgcagcctgatttttggcgtggtggatcatctgaaacagcatctgctgaccgatcatggatccggacctaagaaaaagaggaaggtggcggccgcttacccatacgatgttccagattacgctgacgtggaattctaacaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 29 - DNA, 1938 bp, Artificial Sequence
  ZNF fusion protein
cctgcaggcagctgcgcgctcgctcgctcactgaggccgcccgggcgtcgggcgacctttggtcgcccggcctcagtgagcgagcgagcgcgcagagagggagtgggcggccgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgaccggttctagagcgctgccaccatgagccccaagaagaagagaaaggtggaggccagcatgcagatggagctcgaaatcaggccgctgttcctcgtgccggacactaatggttttatagatcacttggcgtccttggctagacttctggaaagccgaaagtatatattggtagtgccgttgattgtaattaacgaattggatgggttggcgaaaggacaagagactgatcacagagcaggaggctacgcgagggtcgtccaagagaaggcgcgaaaaagcatcgagttcctggagcagcgatttgagagcagggactcatgcctgagagccctcacgtctcgggggaacgagctggagtccatcgctttccgaagtgaagacattacgggccaacttgggaataatgatgacctcatcttgtcctgctgcctgcactactgcaaggacaaggctaaggacttcatgcctgcctccaaggaggagcctatccgattgttgagggaagtagtacttttgacggacgaccgcaacctccgggtaaaggcgctgactcgaaatgtcccagtaagggatataccggcgttccttacatgggctcaagtagggagtggaagtgagacaccgggaacctcagagagcgccacgccagaaagccatgaatgccgcgtgtgcggcgtgaccgaagtgggcctgagcgcgtatgcgaaacatattagcggccagctgcatggcagcggaagtgggtatcgctgctggtggcatggctgcagcctgatttttggcgtggtggatcatctgaaacagcatctgctgaccgatcatggatccggacctaagaaaaagaggaaggtggcggccgcttacccatacgatgttccagattacgctgacgtggaattctaacaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 30 - protein, 16 aa, Artificial Sequence
  Linker
SGSETPGTSESATPES

SEQ ID NO: 31 - protein, 12 aa, Artificial Sequence
  Linker
SGSETPGTSESA

SEQ ID NO: 32 - protein, 21 aa, Artificial Sequence
  Linker
SGSETPGTSESATPEGGSGGS

SEQ ID NO: 33 - protein, 5 aa, Artificial Sequence
  Linker
  REPEAT (1)..(5): sequence may be repeated 1, 2, 3, 4 or 5 times
GGGGS